(19) United States
(12) Patent Application Publication
(10) Pub. No.: US 2021/0261513 A1
(43) Pub. Date: Aug. 26, 2021

(54) 4-SUBSTITUTED PHENYL-1,3,5-TRIAZINE DERIVATIVES AS MODULATORS OF TRK RECEPTORS

(71) Applicant: ALZECURE PHARMA AB, Huddinge (SE)

(72) Inventors: Gunnar NORDVALL, Rönninge (SE); Pontus FORSELL, Huddinge (SE)

(21) Appl. No.: 17/255,632

(22) PCT Filed: Jun. 28, 2019
(86) PCT No.: PCT/GB2019/051852

§ 371 Date: Dec. 23, 2020

(30) Foreign Application Priority Data
Jun. 28, 2018 (GB) 1810669.0

(51) Int. Cl. C07D 251/04; C07D 405/10; C07D 403/10; A61K 31/53

(52) U.S. Cl. CPC C07D 251/04; A61K 31/53; C07D 403/10; C07D 405/10

(57) ABSTRACT

There is provided a compound of formula I, wherein R¹, R² R³, Q¹, Q² and Q³ are as defined herein, which compounds are useful in the treatment of diseases characterised by impaired signalling of neurotrophins and/or other trophic factors, such as Alzheimer's disease and the like.

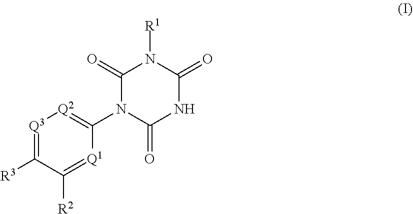

### FIELD OF THE INVENTION

This invention relates to novel pharmaceutically-active compounds, to pharmaceutical compositions comprising such compounds, as well as to their pharmaceutical use. In particular, the invention relates to the use of these compounds and compositions in methods for the treatment and/or prevention of diseases characterised by impaired signalling of neurotrophins and/or other trophic factors.

### BACKGROUND OF THE INVENTION

The listing or discussion of an apparently prior-published document in this specification should not necessarily be taken as an acknowledgement that the document is part of the state of the art or is common general knowledge.

Nerve growth factor (NGF), Brain Derived Neurotrophic Factor (BDNF), neurotrophin-3 (NT-3) and neurotrophin-4/5 all belong to the neurotrophin protein family. These hormones act through a class of receptor tyrosine kinases called tropomyosin-receptor kinase (Trk). Ligand binding to Trks initiates receptor dimerization and autophosphorylation of the kinase domain, which activates the kinase activity of the receptor. This results in further receptor phosphorylation at Tyr490, Tyr751 and Tyr785 of TrkA (or their equivalent residues in other Trk receptors). This phosphorylation leads to adaptor binding sites that couple the receptor to SHC adaptor protein 1 (SHC-1), phosphoinositide 3-kinase (PI3K) and phospholipase Cγ1 (PLCγ1). The coupling of adaptor proteins to the receptor initiates several different cellular events leading to e.g. neurite outgrowth and axonal elongation. These receptors, and their signalling pathways, play a pivotal role in many key processes in the brain e.g. hippocampal neurogenesis, synaptic plasticity, and long-term potentiation, a proposed mechanism underlying memory formation at the level of the synapse. Both NGF/TrkA and BDNF/TrkB-stimulated signalling is also necessary for the survival and morphogenesis of neurons.

In addition to activation of Trk-receptors by classical ligand binding, there are ligand independent events that can regulate neurotrophin signalling.

The balance between the activity of the receptor tyrosine kinase and the activity of tyrosine phosphatases intricately regulates the levels of phosphorylated receptor. Thus, protein tyrosine phosphatases such as PTP-1B or other phosphatases can increase neurotrophin signalling and regulate temporal and spatial activity of the Trk-receptor as well as receptor tyrosine kinases.

Also, adenosine and adenosine agonists can mediate phosphorylation of Trk-receptors, via a mechanism that requires the adenosine 2A (A2A) receptor. This phosphorylation of Trk-receptors is independent of ligand binding suggesting that modulation of Trk-receptor signalling can be accomplished by several different mechanisms.

Synapse loss and a decrease in the hippocampal volume are pathological signatures of Alzheimer's disease in the brain and a number of studies suggest that synapse loss is the best neuroanatomical indicator of cognitive decline in the disease. Basal forebrain cholinergic neurons (BFCN) are a subpopulation of neurons that seem to be particularly vulnerable to the pathology of AD. Dysfunctional atrophy of these neurons, which in turn results in severe loss of cortical and hippocampal innervation, may be the source for the malfunction of the cholinergic system in AD (Bartus RT Exp Neurol 2000; 163:495-529). The severe cortical cholinergic deficits in the disease also include a loss of choline acetyltransferase (ChAT) and acetylcholinesterase (AChE) activity. The basal forebrain cholinergic system is dependent on NGF and cholinergic basal forebrain neurons are the major cell group that expresses the receptor for NGF, i.e. TrkA. Although the role of NGF in cholinergic neuronal survival and function is well established, studies have also shown neuroprotective/neurorestorative effects mediated by this system, e.g. that axotomized cholinergic projections in animals can be rescued by TrkA activation (Lucidi-Phillipi C A, Neuron., 1996, 16(3):653-663).

An early morphological change in the brain of AD-patients is a decreased hippocampal volume. BDNF/TrkB-stimulated signalling has previously been shown to be necessary for survival and morphogenesis of especially hippocampal neurons. Moreover, it is widely accepted that BDNF plays a critical role in neuronal plasticity and long-term potentiation (LTP). Indeed, a growing body of experimental evidence suggests that increased BDNF signalling could potentially improve cognition in AD. The transplantation of stem cells into the brain of a triple-transgenic mouse model of AD, that expresses amyloid and tau pathology, i.e. the major neuropathological hallmarks of AD, results in improved cognition (Blurton-Jones M, PNAS, 2009. 106(32): p. 13594-13599). This effect is mediated by BDNF as gain-of-function studies show that recombinant BDNF mimics the beneficial effects of neural stem cell (NSC) transplantation. Furthermore, loss-of-function studies show that depletion of NSC-derived BDNF fails to improve cognition or restore hippocampal synaptic density.

Given the potent neuroprotective and neurorestorative effects of the TrkA/NGF and TrkB/BDNF systems, small molecule positive modulators of neurotrophin signalling might be beneficial in treating a number of diseases with neurodegeneration including, but not limited to, Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and other parkinsonian disorders. The modulators can also be used in the treatment of diseases where enhancement of nerve regeneration is beneficial, such as demyelinating diseases including, but not limited to, multiple sclerosis. The modulators could also be used for neuroprotection before or after an insult such as spinal cord injury, stroke, hypoxia, ischemia, brain injury including traumatic brain injury. Moreover, the important role of these neurotrophin systems in synaptic plasticity is thought to mediate learning and memory processes, and indicates that the modulators could also be used in disorders where cognitive function is impaired, including, but not limited to, mild cognitive impairment, dementia disorders (including dementia of mixed vascular and degenerative origin, presenile dementia, senile dementia and dementia associated with Parkinson's disease, progressive supranuclear palsy or corticobasal degeneration) and cognitive dysfunction in schizophrenia.

Recent data have also indicated that NGF/TrkA and BDNF/TrkB systems may operate as metabotrophins, that is, be involved in the maintenance of cardiometabolic homeostasis (glucose and lipid metabolism as well as energy balance, cardioprotection, and wound healing) (Chaldakov G, Arch. Ital. Biol. 2011 Jun. 149(2):257-63). In fact, mutations in the genes encoding BDNF and its receptor TrkB have been shown to lead to severe obesity in humans (Yeo, GS. et al. Nat. Neurosci. 2004, 7, 1187-1189). Therefore, indications such as atherosclerosis, obesity, diabetes and metabolic syndrome could also benefit from NGF/TrkA and BDNF/TrkB directed therapies.

Another area of interest when it comes to neurotrophin signalling is neuropsychiatric disorders (Castén E et al., Neurobiol Dis. 2016 Jul. 15, 30169-3). Studies have, for example, clearly demonstrated that depressed patients have reduced serum BDNF levels, which are restored after successful recovery (Shimizu et al., 2003, Sen et al., 2008). Moreover, several studies have demonstrated that chronic treatment with various antidepressant drugs increase BDNF mRNA and protein levels in the cerebral cortex and hippocampus (Calabrese et al., Psychopharmacology, 2011, 215, pp. 267-275). Also, local administration of BDNF into the brain has been shown to reduce depression-like behavior and mimic the effects of antidepressants (Hoshaw et al., Brain Res., 2005, 1037, pp. 204-208). Notably, the role for BDNF does not seem to be restricted to depression; it has also been implicated in other disorders, such as anxiety and schizophrenia (Casten E., Handb. Exp. Pharmacol., 2014, 220, pp. 461-479). These data suggest that therapies targeting neurotrophin systems e.g. NGF/TrkA and BDNF/TrkB could have a therapeutic effect in several neuropsychiatric disorders, including, but not limited to, depression, schizophrenia and anxiety.

It has also been demonstrated that both BDNF and NT3 stimulate gastrointestinal motility, accelerate colonic transit time and relieve constipation in humans (Coulie B., et al., Gastroenterology, 2000, 119(1), 41-50). The levels of BDNF in colonic biopsies from patients with slow-transit constipation have also been found to be reduced compared to healthy controls (Chen et al., Acta. Physiol., 2014, 212(3), 226-238). Furthermore, one of the observed adverse events in a large phase 3 clinical trial with >1,000 patients with the neurological disorder amyotrophic lateral sclerosis undergoing treatment with recombinant BDNF was increased gut motility, diarrhoea and relief of constipation (Neurology, 1999, 52(7), 1427). A similar observation was also made in a smaller clinical trial of BDNF in patients with diabetic neuropathy (VVellmer A. et al., J. Peripher. Nerv. Syst., 2001, 6(4), 204-210). Preclinical models have also suggested that BDNF and NT3 may play a role in the regulation of gastrointestinal motility. BDNF +/−heterozygous mice display decreased stool frequency and increased total gastrointestinal transit time, demonstrating that lower BDNF levels reduces gastrointestinal motility. BDNF also relieved loperamide-induced constipation in mice (Chen et al., Acta. Physiol., 2014, 212(3), 226-238). Accordingly, it is believed that neurotrophins and their receptors may play an important role in maintaining normal motility and therefore that modulation of neurotrophin signaling could represent a promising strategy for improving gut motility in patients suffering from constipation.

The finding that NGF and BDNF play important roles in neuronal homeostasis in combination with their neuroprotective and neurorestorative effect makes these pathways highly suitable as candidates for drug intervention for the treatment of diseases of the central nervous system and the peripheral nervous system. However, BDNF and NGF are themselves not ideal drug candidates due to their pharmacokinetic properties, the difficulties in administration and their limited ability to cross the blood-brain barrier. This has led to several attempts to identify peptides, cyclized peptides, peptide mimetics, small molecule agonist or selective modulators of NGF or BDNF. Several natural products such as gambogic amide (and analogues thereof), deoxygedunin and 7,8-dihydroxyflavone have been demonstrated to act as TrkA or TrkB agonists. Moreover, the tricyclic depressant amitriptyline has also been shown to be a TrkA and TrkB agonist. However, there is currently no specific TrkA or TrkB agonist that has reached the market. Therefore, there is an unmet need in the art for small molecule compounds that have the ability to stimulate or modulate TrkA and/or TrkB receptors, in combination with TrkC, FGFR1 and/or IGF1R and optionally other receptor tyrosine kinases for the treatment of both neurological and non-neurological disorders. There is still a need for compounds that have an improved potency and improved selectivity to TrkA and/or TrkB receptor.

BDNF production can be affected by a polymorphism within the BDNF gene (rs6265) causes a valine (Val) to methionine (Met) substitution at codon 66 (Val66Met). This polymorphism is found in approximately 30% of Caucasians and up to 70% in Asian populations. The presence of one or two Met alleles is associated with lower BDNF production in a subject. This lower BDNF production can lead to increased cognitive decline and decreased hippocampal volume.

A study by Boots et al (Neurology, 2017, 88, 1-9) demonstrated that subjects suffering sporadic Alzheimer's disease who carry the BDNF Met allele experience a steeper decline in episodic memory and executive function than non-carriers. Greater memory decline and decreased hippocampal function have also been observed in Val66Met patients with familial Alzheimer's disease (Lim et al., Brain, 2016, 139(10), 2766-2777). The same study also showed increased tau-protein and phosphorylated tau-protein in the cerebrospinal fluid in this patient group. The decline in memory in subjects with pre-clinical or clinical Alzheimer's disease was exacerbated by greater amyloid plaque burden, thus suggesting that it is possible to treat Alzheimer's disease at various stages of the disease by potentiating the effects of BDNF in patients with the Val66Met polymorphism. Such treatment may lead to neuroprotection and increased cognitive function.

In general therefore, there remains a need for alternative and/or more effective compounds that are useful in the treatment and/or prevention of diseases characterised by impaired signalling of neurotrophins and/or other trophic factors, and in particular neurodegenerative diseases such as Alzheimer's disease.

Toltrazuril (1-methyl-3-(3-methyl-4-{4-[(trifluoromethyl)sulfanyl]phenoxy}phenyl)-1,3,5-triazinane-2,4,6-trione; Baycox®) is a triazine-based antiprotozoal compound that is used in veterinary medicine to treat coccidial infections, such as isosporiasis, toxoplasmosis, neosporosis, and equine protozoal meningoencephalitis.

A recent study by Suzuki et al. (FEBS Open Bio 2016, 6 461-468), reported that toltrazuril inhibits the binding of p-amyloid oligomers to ephrin type-B receptor 2 (EphB2; a receptor understood to play a role memory and learning functions) by 30%. However, due to a lack of selectivity for this receptor, it was not selected for further studies as a potential candidate compound for the treatment of Alzheimer's disease.

Other phenyl-1,3,5-triazine derivatives are disclosed for similar use in veterinary medicine in several old patent documents, such as U.S. Pat. No. 3,933,814, SE 402 103, DE 3 408 768 A1, EP 0 081 142 A2 and 279 219 A1. There is no suggestion that any of the compounds that are disclosed in any of these documents may be used to treat human patients per se and certainly not that the compounds may be useful in the treatment and/or prevention of diseases characterised by impaired signalling of neurotrophins and/or other trophic factors, such as Alzheimer's disease.

It has now surprisingly been found that certain 4-substituted phenyl-1,3,5-triazine derivatives are positive modulators of Trk receptors (including TrkA, TrkB and TrkC) and receptor tyrosine kinases such as IGF1R and/or FGFR1, and thus have properties rendering them useful for the treatment of diseases characterised by impaired signalling of neurotrophins and/or other trophic factors, such as Alzheimer's disease. As a result of their mode of action, the compounds are thought to be particularly suitable as therapeutic agents for use in disorders such as Alzheimer's disease, for example in patients having the Val66Met mutation in the brain-derived neurotrophic factor (BDNF) gene.

### DETAILED DESCRIPTION OF THE INVENTION

According to an aspect of the invention, there is provided a compound of formula I,

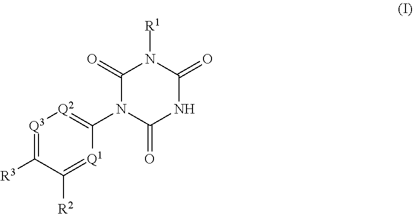

wherein

R¹ represents methyl; phenyl optionally substituted by one or more groups selected from halogen, —CN, —C(O)NR^{a1} R^{a2}, —NRₐ₃R^{a4}, a 5-membered heteroaryl group, C₁₋₄ alkyl, C₁₋₄ alkoxy, C₁₋₄ alkoxy-C₁₋₄ alkyl or methylenedioxy, wherein the latter four groups are optionally substituted by one or more fluoro groups; or a 5-9-membered heteroaryl group optionally substituted by one or more groups selected from halogen, —CN, C₁₋₄ alkyl, C₁₋₄ alkoxy C₁₋₄ alkoxy-C₁₋₄ alkyl or phenyl, wherein the latter four groups groups are optionally substituted by one or more fluoro groups;

Q¹, Q², and Q³ each represent —C(R⁴)— or —N—, wherein a maximum of two of Q¹ to Q³ represent -N-;

R⁴ represents H, halogen, C₁₋₄ alkyl, C₁₋₄ alkoxy or C₁₋₄ alkoxy-C₁₋₄ alkyl, which latter three groups are optionally substituted by one or more fluoro groups;

R² represents halo; cyano; C₁₋₆ alkyl or C₁₋₆ alkoxy, which latter two groups are optionally substituted by one or more fluoro, ═O, hydroxy, —NR^{a5}R^{a6} or C₁₋₂ alkoxy groups;

R³ represents aryl optionally substituted by one or more G¹ groups; a 5-10-membered heteroaryl group optionally substituted by one or more G² groups; each of which two groups (i.e. the optionally substituted aryl or 5-10-membered heteroaryl groups) are optionally linked to the Q¹ to Q³ containing ring via an ethynylene group; a 4-10-membered heterocyclyl group optionally substituted by one or more G³ groups; C₁₋₆ alkyl, C₁₋₆ alkenyl, C₁₋₆ alkynyl, C₁₋₆ alkoxy or —N(C₁₋₆ alkyl)(C₁₋₆ alkyl), which latter five groups are optionally substituted by one or more G⁴ groups;

G¹ represents halogen; hydroxy; cyano; or C₁₋₄ alkyl, C₁₋₄ alkoxy or —NR^{a7}R^{a8}, wherein each of the latter three groups is optionally substituted by one or more fluoro groups;

G² represents halogen; hydroxy; cyano; or C₁₋₄ alkyl, C₁₋₄ alkoxy or —NR^{a9}R^{a10,} wherein each of the latter three groups is optionally substituted by one or more fluoro groups; or phenyl or a 5-9-membered heteroaryl group, each of which two groups is optionally substituted by one or more groups selected from halo, —CN, C₁₋₂ alkyl, C₁₋₂ alkoxy or C₁₋₂ alkoxy-C₁₋₂ alkyl;

G³ represents halogen; hydroxy; cyano; or C₁₋₄ alkyl or C₁₋₄ alkoxy, wherein each of the latter two groups is optionally substituted by one or more fluoro groups; or phenyl or a 5-9-membered heteroaryl group, each of which two groups is optionally substituted by one or more groups selected from halo, —CN, C₁₋₂ alkyl or C₁₋₂ alkoxy;

G⁴ represents halogen, ═O, hydroxy or C₁₋₂ alkoxy;

R^{a1}, R^{a2}, R^{a3}, R^{a4}, R^{a5}, R^{a6}, R^{a7}, R^{a8}, R^{a9} and R^{a10} each independtly represent H or C₁₋₄ alkyl, which C₁₋₄ alkyl groups are optionally substituted by one or more fluoro groups; or

R^{a1} and R^{a2}, R^{a3} and R^{a4}, R^{a5} and R^{a6}, R^{a7} and R^{a8}, R^{a9} and R^{a10} may independently be joined together to form, together with the atom to which they are attached, a 4- to 6-membered heterocyclyl ring, which heterocyclyl ring optionally contains one further heteroatom selected from N, O or S.

or a pharmaceutically-acceptable salt thereof,

which compounds (including pharmaceutically acceptable salts) may be referred to herein as the “compounds of the invention”.

For the avoidance of doubt, the skilled person will understand that references herein to compounds of particular aspects of the invention (such as the first aspect of the invention, i.e. referring to compounds of formula I as defined in the first aspect of the invention) will include references to all embodiments and particular features thereof, which embodiments and particular features may be taken in combination to form further embodiments and features of the invention.

Unless indicated otherwise, all technical and scientific terms used herein will have their common meaning as understood by one of ordinary skill in the art to which this invention pertains.

Pharmaceutically acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared using techniques known to those skilled in the art, such as by exchanging a counter-ion of a compound of the invention in the form of a salt with another counter-ion, for example using a suitable ion exchange resin.

Particular acid addition salts that may be mentioned include those formed by reaction with corresponding acids, thus protonating the compound of the invention, to form carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, a-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxy-benzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxy-ethanesulphonate, 1- or 2-naphthalene-sulphonate or 1,5-naphthalene-disulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed by reaction with corresponding bases, thus removing a proton from compounds of the invention, to form salts with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

More particular salts that may be mentioned include Na salts.

For the avoidance of doubt, compounds of the invention may exist as solids, and thus the scope of the invention includes all amorphous, crystalline and part crystalline forms thereof, and may also exist as oils. Where compounds of the invention exist in crystalline and part crystalline forms, such forms may include solvates, which are included in the scope of the invention.

For the avoidance of doubt, compounds of the invention may also exist in solution (i.e. in solution in a suitable solvent). For example, compounds of the invention may exist in aqueous solution, in which case compounds of the invention may exist in the form of hydrates thereof.

Compounds of the invention may also exhibit tautomerism. All tautomeric forms and mixtures thereof are included within the scope of the invention (particularly those of sufficient stability to allow for isolation thereof).

Compounds of the invention may also contain one or more asymmetric carbon atoms and may therefore exhibit optical and/or diastereoisomerism (i.e. existing in enantiomeric or diastereomeric forms). Diastereoisomers may be separated using conventional techniques, e.g. chromatography or fractional crystallisation. The various stereoisomers (i.e. enantiomers) may be isolated by separation of a racemic or other mixture of the compounds using conventional, e.g. fractional crystallisation or HPLC, techniques. Alternatively the desired enantiomer or diastereoisomer may be obtained from appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution; for example, with a homochiral acid followed by separation of the diastereomeric derivatives by conventional means such as chromatography), or by reaction with an appropriate chiral reagent or chiral catalyst, all of which methods and processes may be performed under conditions known to the skilled person. Unless otherwise specified, all stereoisomers and mixtures thereof are included within the scope of the invention.

For the avoidance of doubt, the skilled person will understand that where a particular group is depicted herein as being bound to a ring system via a floating bond (i.e. a bond not shown as being bound to a particular atom within the ring), the relevant group may be bound to any suitable atom within the relevant ring system (i.e. the ring within which the floating bond terminates).

Unless otherwise specified, C_{1-z} alkyl groups (where z is the upper limit of the range) defined herein may be straight-chain or, when there is a sufficient number (i.e. a minimum of two or three, as appropriate) of carbon atoms, be branched-chain, and/or cyclic (so forming a C_{3-z} cycloalkyl group). When there is a sufficient number (i.e. a minimum of four) of carbon atoms, such groups may also be part cyclic (so forming a C_{4-z} partial cycloalkyl group). For example, cycloalkyl groups that may be mentioned include cyclopropyl, cyclopentyl and cyclohexyl. Similarly, part cyclic alkyl groups (which may also be referred to as “part cycloalkyl” groups) that may be mentioned include cyclopropylmethyl. When there is a sufficient number of carbon atoms, such groups may also be multicyclic (e.g. bicyclic or tricyclic) and/or spirocyclic. For the avoidance of doubt, particular alkyl groups that may be mentioned include straight chain (i.e. not branched and/or cyclic) alkyl groups.

Unless otherwise specified, C_{1-z} alkoxy groups (i.e. —OC_{1-z} alkyl groups) (where z is the upper limit of the range) defined herein may be straight-chain or, when there is a sufficient number (i.e. a minimum of two or three, as appropriate) of carbon atoms, be branched-chain, and/or cyclic (so forming a —OC_{3-z} cycloalkoxy group). When there is a sufficient number (i.e. a minimum of four) of carbon atoms, such groups may also be part cyclic (so forming a —OC_{4-z} partial cycloalkoxy group). For example, cycloalkyloxy groups that may be mentioned include cyclopropoxy, cyclopentoxy and cyclohexoxy. Similarly, part cyclic alkoxy groups (which may also be referred to as “part cycloalkoxy” groups) that may be mentioned include cyclopropylmethoxy. When there is a sufficient number of carbon atoms, such groups may also be multicyclic (e.g. bicyclic or tricyclic) and/or spirocyclic. For the avoidance of doubt, particular alkoxy groups that may be mentioned include straight chain (i.e. not branched and/or cyclic) alkoxy groups.

For the avoidance of doubt, references to C_{1-z} alkoxy-C_{1-z} alkyl groups indicate an alkoxyalkyl group (i.e. —C_{1-z} alkyl-O—C_{1-z} alkyl groups) such as methoxymethyl groups. As for alkyl and alkoxy groups, unless otherwise specified such groups may be straight-chain, or when there is a sufficient number of carbon atoms, be branched chain, cyclic and/or part cyclic.

Unless otherwise specified, C_{2-z} alkenyl groups (where z is the upper limit of the range) defined herein may be straight-chain or, when there is a sufficient number (i.e. a minimum of three) of carbon atoms, be branched-chain, and/or cyclic (so forming a C_{4-z} cycloalkenyl group). When there is a sufficient number (i.e. a minimum of five) of carbon atoms, such groups may also be part cyclic. For example, part cyclic alkenyl groups (which may also be referred to as “part cycloalkenyl” groups) that may be mentioned include cyclopentenylmethyl and cyclohexenylmethyl. When there is a sufficient number of carbon atoms, such groups may also be multicyclic (e.g. bicyclic or tricyclic) or spirocyclic. For the avoidance of doubt, particular alkenyl groups that may be mentioned include straight chain (i.e. not branched and/or cyclic) alkenyl groups. C_{2-z} alkenyl groups may contain one or more double bonds. Particular C_{2-z} alkenyl groups that may be mentioned include those containing one double bond.

Unless otherwise specified, C_{2-z} alkynyl groups (where z is the upper limit of the range) defined herein may be straight-chain or, when there is a sufficient number (i.e. a minimum of four) of carbon atoms, be branched-chain. For the avoidance of doubt, particular alkynyl groups that may be mentioned include straight chain (i.e. not branched and/or cyclic) alkynyl groups. C_{2-z} alkynyl groups may contain one or more double bonds. Particular C_{2-z}alkynyl groups that may be mentioned include those containing one double bond.

For the avoidance of doubt, unless otherwise specified, groups referred to herein as “alkyl”, “alkenyl” and/or “alkynyl” will be taken as referring to the highest degree of unsaturation in a bond present in such groups. For example, such a group having a carbon-carbon double bond and, in the same group, a carbon-carbon triple bond will be referred to as “alkynyl”. Alternatively, it may be particularly specified that that such groups will comprise only the degree of unsaturation specified (i.e. in one or more bond therein, as appropriate; e.g. in in one bond therein).

For the avoidance of doubt, alkyl, alkenyl and alkynyl groups as described herein may also act as linker groups (i.e. groups joining two or more parts of the compound as described), in which case such groups may be referred to as “alkylene”, “alkenylene” and/or “alkynylene” groups, respectively.

As used herein, the term heterocyclyl may refer to non-aromatic monocyclic and polycyclic (e.g. bicyclic) heterocyclic groups (which groups may, where containing a sufficient number of atoms, also be bridged) in which at least one (e.g. one to four) of the atoms in the ring system is other than carbon (i.e. a heteroatom), and in which the total number of atoms in the ring system is between three and twelve (e.g. between five and ten, such as between three and eight; for example, forming a 5- or 6-membered heterocyclyl group).

Further, such heterocyclyl groups may be saturated, forming a heterocycloalkyl, or unsaturated containing one or more carbon-carbon or, where possible, carbon-heteroatom or heteroatom-heteroatom double and/or triple bonds, forming for example a C_{2-z} (e.g. C_{4-z}) heterocycloalkenyl (where z is the upper limit of the range) or a C_{7-z} heterocycloalkynyl group.

For the avoidance of doubt, the skilled person will understand that heterocyclyl groups that may form part of compounds of the invention are those that are chemically obtainable, as known to those skilled in the art. Various heterocyclyl groups will be well-known to those skilled in the art, such as 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, 2,3-dihydroisothiazolyl, dihydropyranyl, dihydropyridinyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, isothiazolidinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuryl, tetrahydropyridinyl (such as 1,2,3,4-tetrahydropyridinyl and 1,2,3,6-tetrahydropyridinyl), thietanyl, thiiranyl, thiolanyl, tetrahydrothiopyranyl, thiomorpholinyl, trithianyl (including 1,3,5-trithianyl), tropanyl and the like.

Substituents on heterocyclyl groups may, where appropriate, be located on any atom in the ring system including a heteroatom. Further, in the case where the substituent is another cyclic compound, then the cyclic compound may be attached through a single atom on the heterocyclyl group, forming a spirocyclic compound. The point of attachment of heterocyclyl groups may be via any suitable atom in the ring system, including (where appropriate) a further heteroatom (such as a nitrogen atom), or an atom on any fused carbocyclic ring that may be present as part of the ring system. Heterocyclyl groups may also be in the N— or S-oxidised forms, as known to those skilled in the art.

At each occurrence when mentioned herein, particular heterocyclyl groups that may be mentioned include 4- to 8-membered heterocyclyl groups (e.g. a 4- to 6-membered heterocyclyl group, such as a 5- or 6-membered heterocyclyl group).

For the avoidance of doubt, references to polycyclic (e.g. bicyclic or tricyclic) groups (for example when employed in the context of heterocyclyl or cycloalkyl groups (e.g. heterocyclyl)) will refer to ring systems wherein at least two scissions would be required to convert such rings into a non-cyclic (i.e. straight or branched) chain, with the minimum number of such scissions corresponding to the number of rings defined (e.g. the term bicyclic may indicate that a minimum of two scissions would be required to convert the rings into a straight chain). For the avoidance of doubt, the term bicyclic (e.g. when employed in the context of alkyl groups) may refer to groups in which the second ring of a two-ring system is formed between two adjacent atoms of the first ring, to groups in which two non-adjacent atoms are linked by an alkyl (which, when linking two moieties, may be referred to as alkylene) group (optionally containing one or more heteroatoms), which later groups may be referred to as bridged, or to groups in which the second ring is attached to a single atom, which latter groups may be referred to as spiro compounds.

Particular heterocyclyl groups that may be mentioned include piperidinyl (e.g. piperidin-1-yl), octahydro-1H-isoindolyl (e.g. octahydro-1H-isoindo1-2-yl), azetidinyl (e.g. azetidine-1-yl), oxetanyl (e.g. oxetan-3-yl), morpholinyl (e.g. morpholin-4-yl), piperazinyl (e.g. piperazin-1yl or piperazin-4-yl), azepanyl (e.g. azepan-1-yl), imidazolidinyl (e.g. imidazolidine-2-yl), pyrrolidinyl (e.g. pyrrolidine-1yl), and diazepanyl (e.g. 1,4-diazepan-1-yl).

As may be used herein, the term aryl may refer to C₁₋₁₄ (e.g. C₆₋₁₀) aromatic groups. Such groups may be monocyclic or bicyclic and, when bicyclic, be either wholly or partly aromatic. C₆₋₁₀ aryl groups that may be mentioned include phenyl, naphthyl, 1,2,3,4-tetrahydronaphthyl, indanyl, and the like (e.g. phenyl, naphthyl, and the like). For the avoidance of doubt, the point of attachment of substituents on aryl groups may be via any suitable carbon atom of the ring system.

For the avoidance of doubt, the skilled person will understand that aryl groups that may form part of compounds of the invention are those that are chemically obtainable, as known to those skilled in the art. Particular aryl groups that may be mentioned include phenyl and naphthyl, such as phenyl.

As may be used herein, references to heteroaryl (with may also be referred to as heteroaromatic) groups may refer to 5- to 14- (e.g. 5- to 10-) membered heteroaromatic groups containing one or more heteroatoms (such as one or more heteroatoms selected from oxygen, nitrogen and/or sulfur). Such heteroaryl groups may comprise one, two, or three rings, of which at least one is aromatic. Substituents on heteroaryl/heteroaromatic groups may, where appropriate, be located on any suitable atom in the ring system, including a heteroatom (e.g. on a suitable N atom).

The point of attachment of heteroaryl/heteroaromatic groups may be via any atom in the ring system including (where appropriate) a heteroatom. Bicyclic heteroaryl/heteroaromatic groups may comprise a benzene ring fused to one or more further aromatic or non-aromatic heterocyclic rings, in which instances, the point of attachment of the polycyclic heteroaryl/heteroaromatic group may be via any ring including the benzene ring or the heteroaryl/heteroaromatic or heterocyclyl ring.

For the avoidance of doubt, the skilled person will understand that heteroaryl groups that may form part of compounds of the invention are those that are chemically obtainable, as known to those skilled in the art. Various heteroaryl groups will be well-known to those skilled in the art, such as pyridinyl, pyrrolyl, furanyl, thiophenyl, oxadiazolyl, thiadiazolyl, thiazolyl, oxazolyl, pyrazolyl, triazolyl, tetrazolyl, isoxazolyl, isothiazolyl, imidazolyl, imidazopyrimidinyl, imidazothiazolyl, thienothiophenyl, pyrimidinyl, furopyridinyl, indolyl, azaindolyl, pyrazinyl, pyrazolopyrimidinyl, indazolyl, pyrimidinyl, quinolinyl, isoquinolinyl, quinazolinyl, benzofuranyl, benzothiophenyl, benzoimidazolyl, benzoxazolyl, benzothiazolyl, benzotriazolyl and purinyl.

For the avoidance of doubt, the oxides of heteroaryl/heteroaromatic groups are also embraced within the scope of the invention (e.g. the N-oxide).

As stated above, heteroaryl includes polycyclic (e.g. bicyclic) groups in which one ring is aromatic (and the other may or may not be aromatic). Hence, other heteroaryl groups that may be mentioned include groups such as benzo[1,3]dioxolyl, benzo[1,4]dioxinyl, dihydrobenzo[d]isothiazole, 3,4-dihydrobenz[1,4]oxazinyl, dihydrobenzothiophenyl, indolinyl, 5H,6H,7H-pyrrolo[1,2-b]pyrimidinyl, 1,2,3,4-tetrahydroquinolinyl, thiochromanyl and the like.

For the avoidance of doubt, where a ring is depicted as having circle therein, its presence shall indicate that the relevant ring is aromatic. Alternatively, aromatic groups may be depicted as cyclic groups comprising therein a suitable number of double bonds to allow for aromaticity.

The present invention also embraces isotopically-labelled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the compounds of the invention also include deuterated compounds, i.e. compounds of the invention in which one or more hydrogen atoms are replaced by the hydrogen isotope deuterium.

For the avoidance of doubt, in cases in which the identity of two or more substituents in a compound of the invention may be the same, the actual identities of the respective substituents are not in any way interdependent. For example, in the situation in which two or more G¹ groups are present, those G¹ groups may be the same or different. Similarly, where two or more G¹ groups are present and each represent C₁₋₂ alkyl optionally substituted by one or more fluoro groups, these groups may also be the same or different.

Further for the avoidance of doubt, when it is specified that a substituent is itself optionally substituted by one or more substituents (e.g. aryl optionally substituted by one or more G¹ groups), these substituents where possible may be positioned on the same or different atoms. Such optional substituents may be present in any suitable number thereof (e.g. the relevant group may be substituted with one or more such substituents, such as one such substituent).

For the avoidance of doubt, where groups are referred to herein as being optionally substituted it is specifically contemplated that such optional substituents may be not present (i.e. references to such optional substituents may be removed), in which case the optionally substituted group may be referred to as being unsubstituted.

For the avoidance of doubt, the skilled person will appreciate that compounds of the invention that are the subject of this invention include those that are obtainable, i.e. those that may be prepared in a stable form. That is, compounds of the invention include those that are sufficiently robust to survive isolation, e.g. from a reaction mixture, to a useful degree of purity.

Compounds of the invention that may be mentioned include those in which R¹ represents methyl; phenyl optionally substituted by one or more (e.g. one) groups selected from halogen, —CN, —C(O)NR^{a1}R^{a2}, —NR^{a3}R^{a4}, a 5-membered heteroaryl group (e.g. pyrrolyl or pyrazolyl), C₁₋₄ alkyl, C₁₋₄ alkoxy, C₁₋₄ alkoxy-C₁₋₄ alkyl or methylenedioxy, wherein the latter four groups are optionally substituted by one or more (e.g.one) fluoro groups; or a 5-9-membered heteroaryl group (e.g. thiophenyl, pyrazolyl, thiazolyl, pyridinyl, benzofuranyl or indolyl) optionally substituted by one or more (e.g. one) groups selected from halogen, —CN, C₁₋₄ alkyl, C₁₋₄ alkoxy, C₁₋₄ alkoxy-C₁₋₄ alkyl or phenyl, which latter four groups are optionally substituted by one or more fluoro groups, wherein R^{a1}, R^{a2}, R^{a3} and R^{a4} are as defined herein,

Compounds of the invention that may be mentioned include those in which R¹ represents phenyl optionally substituted by one or more (e.g. one) groups selected from halogen, —CN, —C(O)NR^{a1}R^{a2}, —NR^{a3}R^{a4}, a 5-membered heteroaryl group (e.g. pyrrolyl or pyrazolyl), C₁₋₄ alkyl, C₁₋₄ alkoxy, C₁₋₄ alkoxy-C₁₋₄ alkyl or methylenedioxy, wherein the latter four groups are optionally substituted by one or more (e.g. one) fluoro groups; or a 5-9-membered heteroaryl group (e.g. thiophenyl, pyrazolyl, thiazolyl, pyridinyl, benzofuranyl or indolyl) optionally substituted by one or more (e.g. one) groups selected from halogen, —CN, C₁₋₄ alkyl, C₁₋₄ alkoxy or phenyl, which latter three groups are optionally substituted by one or more (e.g. one) fluoro groups, wherein R^{a1}R^{a2}, R^{a3} and R^{a4} are as defined herein.

Compounds of the invention that may be mentioned include those in which R¹ represents methyl; phenyl optionally substituted by one or more (e.g. one) groups selected from halogen, —CN, —C(O)NR^{a1}R^{a2}, —NR^{a3}R^{a4}, C₁₋₄ alkyl, C₁₋₄ alkoxy-C₁₋₄ alkyl or methylenedioxy, wherein the latter four groups are optionally substituted by one or more (e.g. one) fluoro groups; or a 5-9-membered heteroaryl group (e.g. thiophenyl, pyrazolyl, thiazolyl, pyridinyl, benzofuranyl or indolyl) optionally substituted by one or more (e.g. one) groups selected from halogen, —CN, C₁₋₄ alkyl, C₁₋₄ alkoxy or phenyl, which latter three groups are optionally substituted by one or more (e.g. one) fluoro groups, wherein Rₐ₁, R^{a2}, R^{a3} and R^{a4} are as defined herein.

Compounds of the invention that may be mentioned include those in which R¹ represents phenyl optionally substituted by one or more (e.g. one) groups selected from halogen, —CN, —C(O)NR^{a1}R^{a2}, —NR^{a3}R^{a4}, C₁₋₄ alkyl, C₁₋₄ alkoxy, C₁₋₄ alkoxy-C₁₋₄ alkyl or methylenedioxy, wherein the latter four groups are optionally substituted by one or more (e.g. one) fluoro groups; or a 5-9-membered heteroaryl group (e.g. thiophenyl, pyrazolyl, thiazolyl, pyridinyl, benzofuranyl or indolyl) optionally substituted by one or more (e.g. one) groups selected from halogen, —CN, C₁₋₄ alkyl, C₁₋₄ alkoxy or phenyl, which latter three groups are optionally substituted by one or more (e.g. one) fluoro groups, wherein R^{a1}, R^{a2}, R^{a3} and R^{a4} are as defined herein.

Compounds of the invention that may be mentioned include those in which R¹ represents methyl; or, preferably, phenyl optionally substituted by one or more (e.g. one) groups selected from halogen, —CN, —C(O)NR^{a1} R^{a2}, —NR^{a3}R^{a4}, C₁₋₄ alkyl (e.g. C₁₋₂ alkyl), C₁₋₄ alkoxy (e.g. (C₁₋₂ alkoxy), C₁₋₄ alkoxy-C₁₋₄ alkyl (e.g. C₁₋₂ alkoxy-C₁₋₂ alkyl) or methylenedioxy, wherein the latter four groups are optionally substituted by one or more (e.g. one) fluoro groups.

Compounds of the invention that may be mentioned include those in which R¹ represents methyl; phenyl optionally substituted by one or more (e.g. one) groups selected from halogen, C₁₋₄ alkyl, C₁₋₄ alkoxy, or methylenedioxy wherein the latter three groups are optionally substituted by one or more (e.g. one) fluoro groups; or a 5-9-membered heteroaryl group optionally substituted by one or more (e.g. one) groups selected from C₁₋₄ alkyl, C₁₋₄ alkoxy and phenyl, each of which groups is optionally substituted by one or more fluoro groups.Compounds of the invention that may be mentioned include those in which R¹ represents methyl; phenyl optionally substituted by one or more (e.g. one) groups selected from fluoro, chloro, bromo, C₁₋₂ alkyl, C₁₋₂ alkoxy and methylenedioxy, wherein the latter three groups are optionally substituted by one or more (e.g. one) fluoro groups, thiophenyl, thiazolyl, pyrazolyl, pyridinyl, benzofuranyl or indolyl each of which is optionally substituted by one or more groups selected from C₁₋₂ alkyl, C₁₋₂ alkoxy and phenyl each of which may be optionally substituted by one or more (e.g. one) fluoro groups.

Compounds of the invention that may be mentioned include those in which R¹ represents phenyl optionally substituted by one or more (e.g. one) groups selected from fluoro, chloro, bromo, C₁₋₂ alkyl, C₁₋₂ alkoxy and methylenedioxy, wherein the latter three groups are optionally substituted by one or more (e.g. one) fluoro groups; thiophenyl, thiazolyl, pyrazolyl, pyridinyl, benzofuranyl or indolyl each of which is optionally substituted by one or more groups selected from C₁₋₂ alkyl, C₁₋₂ alkoxy and phenyl each of which may be optionally substituted by one or more (e.g. one) fluoro groups.

Compounds of the invention that may be mentioned include those in which R¹ represents methyl; phenyl optionally substituted by one or more (e.g. one) groups selected from fluoro, chloro, bromo, C₁₋₂ alkyl, C₁₋₂ alkoxy, wherein the latter two groups are optionally substituted by one or more (e.g. one) fluoro groups, thiophenyl, thiazolyl, pyrazolyl, pyridinyl, benzofuranyl or indolyl each of which is optionally substituted by one or more groups selected from C₁₋₂ alkyl, C₁₋₂ alkoxy and phenyl each of which may be optionally substituted by one or more (e.g. one) fluoro groups.

Compounds of the invention that may be mentioned include those in which R¹ represents phenyl optionally substituted by one or more (e.g. one) groups selected from fluoro, chloro, bromo, C₁₋₂ alkyl, C₁₋₂ alkoxy, wherein the latter two groups are optionally substituted by one or more (e.g. one) fluoro groups, thiophenyl, thiazolyl, pyrazolyl, pyridinyl, benzofuranyl or indolyl each of which is optionally substituted by one or more groups selected from C₁₋₂ alkyl, C₁₋₂ alkoxy and phenyl each of which may be optionally substituted by one or more (e.g. one) fluoro groups

Compounds of the invention that may be mentioned include those in which R¹ represents methyl; phenyl optionally substituted by one or more (e.g. one) groups selected from fluoro, chloro, bromo, C₁₋₂ alkyl, C₁₋₂ alkoxy, wherein the latter two groups are optionally substituted by one or more (e.g. one) fluoro groups; thiophenyl, thiazolyl, pyrazolyl or pyridinyl each of which is optionally substituted by one or more groups selected from C₁₋₂ alkyl (e.g. methyl), C₁₋₂ alkoxy (e.g. methoxy) and phenyl; benzofuranyl or indolyl each of lo which is optionally substituted by one or more groups selected from C₁₋₂ alkyl (e.g methyl) and C₁₋₂ alkoxy (e.g. methoxy).

Compounds of the invention that may be mentioned include those in which R¹ represents methyl; phenyl optionally substituted by one or more (e.g. one) groups selected from fluoro, chloro, bromo, C₁₋₂ alkyl, C₁₋₂ alkoxy, wherein the latter two groups are optionally substituted by one or more F (e.g. unsubstituted), thiophenyl optionally substituted by one or more (e.g. one) methyl groups; pyrazolyl optionally substituted by one or more (e.g. one) methyl or phenyl groups; thiazolyl, pyridinyl, benzofuranyl or indolyl.

Compounds of the invention that may be mentioned include those in which R¹ represents phenyl optionally substituted by one or more (e.g. one) groups selected from fluoro, chloro, bromo, C₁₋₂ alkyl, C₁₋₂ alkoxy, wherein the latter two groups are optionally substituted by one or more F (e.g. unsubstituted), thiophenyl optionally substituted by one or more (e.g. one) methyl groups; pyrazolyl optionally substituted by one or more (e.g. one) methyl or phenyl groups; thiazolyl, pyridinyl, benzofuranyl or indolyl.

Other compounds of the invention that may be mentioned include those in which R¹ represents methyl; phenyl optionally substituted by one or more (e.g. one) groups selected from fluoro, chloro, methyl, ethyl, methoxy, ethoxy, —CF₃, —OCF₃ or methylenedioxy; thiophenyl optionally substituted by one or more (e.g. one) methyl group (e.g. thiophen-2-yl, thiophen-3-yl or 5-methylthiophen-2-yl); pyrazolyl optionally substituted by one or more (e.g. one) methyl or phenyl groups (e.g. pyrazol-3-yl, pyrazol-4-yl, pyrazol-5-yl or, particularly, 1-phenylpyrazol-4-yl); thiazolyl (e.g. thiazol-2-yl, thiazol-4-yl or thiazol-5-yl); pyridinyl (e.g. pyridine-2-yl, pyridine-3-yl or pyridin-4-yl); benzofuranyl (e.g. benzofuran-5-yl; benzofuran-4-yl); or indolyl (e.g. indol-5-yl or indol-4-yl).

Other compounds of the invention that may be mentioned include those in which R¹ represents phenyl optionally substituted by one or more (e.g. one) groups selected from fluoro, chloro, methyl, ethyl, methoxy, ethoxy, —CF₃, —OCF₃ or methylenedioxy; thiophenyl optionally substituted by one or more (e.g. one) methyl group (e.g. thiophen-2-yl, thiophen-3-yl or 5-methylthiophen-2-yl); pyrazolyl optionally substituted by one or more (e.g. one) methyl or phenyl groups (e.g. pyrazol-3-yl, pyrazol-4-yl, pyrazol-5-yl or, particularly, 1-phenyl pyrazol-4-yl); thiazolyl (e.g. thiazol-2-yl, thiazol-4-yl or thiazol-5-yl); pyridinyl (e.g. pyridine-2-yl, pyridine-3-yl or pyridin-4-yl); benzofuranyl (e.g. benzofuran-5-yl; benzofuran-4-yl); or indolyl (e.g. indol-5-yl or indol-4-yl).

Compounds of the invention that may be mentioned include those in which R¹ represents methyl; or phenyl optionally substituted by one group selected from methyl, methoxy, chloro, fluoro, —OCF₃ and methylenedioxy.

Compounds of the invention that may be mentioned include those in which R¹ represents phenyl optionally substituted by one group selected from methyl, methoxy, chloro, fluoro, —OCF₃ and methylenedioxy.

Further compounds of the invention that may be mentioned include those in which R¹ represents methyl, phenyl or tolyl (o-tolyl, p-tolyl or, preferably, m-tolyl).

For the avoidance of doubt, the terms o-tolyl, m-tolyl and p-tolyl may be understood to refer to 2-methylphenyl, 3-methylphenyl and 4-methylphenyl groups, respectively.

Further compounds of the invention that may be mentioned include those in which R¹ represents phenyl or tolyl (o-tolyl, p-tolyl or, preferably, m-tolyl).

Further compounds of the invention that may be mentioned include those in which R¹ represents methyl, phenyl, m-tolyl or p-tolyl.

Further compounds that may be mentioned include those in which R¹ represents phenyl or tolyl (e.g. m-tolyl).

Further compounds of the invention that may be mentioned include those in which R¹ represents methyl or phenyl (particularly phenyl).

Compounds of the invention that may be mentioned include those in which R² represents halo; cyano; C₁₋₆ alkyl (e.g. C₁₋₂ alkyl) or C₁₋₆ alkoxy (e.g. C₁₋₂ alkoxy), which latter two groups are optionally substituted by one or more (e.g. one) fluoro, ═O, hydroxy, alkyl)₂ or C₁₋₂ alkoxy groups.

Compounds of the invention that may be mentioned include those in which R² represents chloro, bromo, C₁₋₄ alkyl (e.g. C₁₋₂ alkyl) or C₁₋₄ alkoxy (e.g. C₁₋₂ alkoxy), which latter two groups are optionally substituted by one or more fluoro, ═O, hydroxy, —NMe₂ or methoxy groups.

Other compounds of the invention that may be mentioned include those in which R² represents C₁₋₂ alkyl optionally substituted by one or more fluoro or methoxy groups.

Other compounds of the invention that may be mentioned include those in which R² represents C₁₋₂ alkyl optionally substituted by one or more fluoro groups.

Further compounds of the invention that may be mentioned include those in which R² represents methyl, ethyl, or —CF₃.

Further compounds of the invention that may be mentioned include those in which R² represents methyl.

Compounds of the invention that may be mentioned include those in which R³ represents phenyl optionally substituted by one or more (e.g. one) G¹ groups; a 5-9-membered heteroaryl group (e.g. a 6-membered heteroaryl group, a 9-membered heteroaryl group or, particularly a 5-membered heteroaryl group) optionally substituted by one or more (e.g. one) G² groups; each of which two groups is optionally linked to the Q¹ to Q³-containing ring via an ethynylene group; a 4-7-membered heterocyclyl group (e.g. a 4-membered heterocyclyl group or a 5-membered heterocyclyl group or, particularly, a 6-membered heterocyclyl group) optionally substituted by one or more (e.g. one) G³ groups; C₁₋₄ alkyl, C₁₋₄ alkenyl, C₁₋₄ alkynyl, C₁₋₄ alkoxy or —N(C₁₋₄ alkyl)(C₁₋₄ alkyl), which latter five groups are optionally substituted by one or more (e.g. one) G⁴ groups, wherein the G¹, G², G³ and G⁴ groups are as defined herein.

For the avoidance of doubt, the term ethynylene group refers to a disubstituted C₂ alkynyl group (i.e.

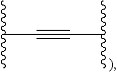

wherein [symbol] represents a point of attachment to the rest of the molecule.

Compounds of the invention that may be mentioned include those in which R³ represents phenyl optionally substituted by one or more (e.g. one) G¹ groups; a 5-9-membered heteroaryl group (e.g. a 6-membered heteroaryl group, a 9-membered heteroaryl group or, particularly a 5-membered heteroaryl group) optionally substituted by one or more (e.g. one) G² groups; a 4-7-membered heterocyclyl group (e.g. a 4-membered heterocyclyl group or a 5-membered heterocyclyl group or, particularly, a 6-membered heterocyclyl group) optionally substituted by one or more (e.g. one) G³ groups; C₁₋₄ alkyl, C₁₋₄ alkenyl, C₁₋₄ alkynyl, C₁₋₄ alkoxy or —N(C₁₋₄ alkyl)(C₁₋₄ alkyl), which latter five groups are optionally substituted by one or more (e.g. one) G⁴ groups, wherein the G¹, G², G³ and G⁴ groups are as defined herein.

Compounds of the invention that may be mentioned include those in which R³ represents phenyl optionally substituted by one or more (e.g. one) G¹ groups or a 5-9-membered heteroaryl group (e.g. a 6-membered heteroaryl group, a 9-membered heteroaryl group or, particularly a 5-membered heteroaryl group) optionally substituted by one or more (e.g. one) G² groups, wherein the G¹ and G² groups are as defined herein.

Other compounds of the invention that may be mentioned include those in which R³ represents phenyl optionally substituted by one or more (e.g one) G¹ groups; a 5-9-membered heteroaryl group selected from pyrrolyl (e.g. pyrol-1-yl), pyrazolyl (e.g. pyrazol-1-yl), imidazolyl (e.g. imidazol-1-yl), triazolyl (e.g. triazol-1-yl), tetrazolyl (e.g. tetrazol-1-yl), oxazolyl, oxadiazolyl, thiazolyl, thiadiazolyl, thiophenyl, furanyl (e.g. furan-2-yl), pyridinyl, pyridazinyl, pyrimidinyl, pyrazinyl, triazinyl, indolyl (e.g. indol-1-yl), benzimidazolyl, benzofuranyl, benzothiophenyl, azaindolyl and azabenzofuranyl (particularly pyrrolyl (e.g. pyrol-1-yl), pyrazolyl (e.g. pyrazol-1-yl) and furanyl (e.g. furan-2-yl)), each of which heteroaryl groups is optionally substituted by one or more (e.g. one) G² groups; each of which phenyl and heteroaryl groups is optionally linked to the Q¹ to Q³-containing ring via an ethynylene group; a 4-6-membered heterocyclyl group selected from azetidinyl (e.g. azetidin-1-yl), piperidinyl (e.g. piperidin-1-yl), piperizinyl (e.g. piperizin-1-yl), morpholinyl (e.g. morpholin-4-yl), pyrrolidinyl (e.g. pyrollidin-1-yl), pyrazolidinyl (e.g. pyrazolidin-1-yl), imidazolidinyl (e.g. imidazolidin-1-yl), imidazolinyl (e.g. imidazolin-1-yl) (particularly piperidinyl (e.g. piperidin-1-yl)), each of which 4-6-membered heterocyclyl groups is optionally substituted by one or more (e.g one) G³ groups; C₁₋₄ alkyl (e.g. methyl, ethyl, propyl, butyl, 2-butyl, tent-butyl or, particularly, iso-propyl) or C₁₋₄ alkoxy (e.g. —Omethyl, —Oethyl, —Opropyl, —Obutyl, —O2-butyl, —Otert-butyl or, particularly, —Oiso-propyl) or —N(C₁₋₄ alkyl)(C₁₋₄ alkyl) (e.g. —N(methyl)₂, —N(ethyl)₂, —N(methyl)(ethyl), —N(propyl)₂, —N(butyl)₂ (particularly —N(methyl)₂)) which latter three groups are optionally substituted by one or more (e.g. one) G⁴ groups, wherein the G¹, G², G³ and G⁴ groups are as defined herein.

Other compounds of the invention that may be mentioned include those in which R³ represents phenyl optionally substituted by one or more (e.g one) G¹ groups; a 5-9-membered heteroaryl group selected from pyrrolyl (e.g. pyrol-1-yl), pyrazolyl (e.g. pyrazol-1-yl), imidazolyl (e.g. imidazol-1-yl), triazolyl (e.g. triazol-1-yl), tetrazolyl (e.g. tetrazol-1-yl), oxazolyl, oxadiazolyl, thiazolyl, thiadiazolyl, thiophenyl, furanyl (e.g. furan-2-yl), pyridinyl, pyridazinyl, pyrimidinyl, pyrazinyl, triazinyl, indolyl (e.g. indol-1-yl), benzimidazolyl, benzofuranyl, benzothiophenyl, azaindolyl and azabenzofuranyl (particularly pyrrolyl (e.g. pyrol-1-yl), pyrazolyl (e.g. pyrazol-1-yl) and furanyl (e.g. furan-2-yl)), each of which heteroaryl groups is optionally substituted by one or more (e.g. one) G² groups; each of which phenyl and heteroaryl groups is optionally linked to the Q¹ to Q³-containing ring via an ethynylene group, wherein G^{l}and G² are as defined herein.

Other compounds of the invention that may be mentioned include those in which R³ represents phenyl optionally substituted by one or more (e.g one) G¹ groups; a 5-9-membered heteroaryl group selected from pyrrolyl (e.g. pyrol-1-yl), pyrazolyl (e.g. pyrazol-1-yl), imidazolyl (e.g. imidazol-1-yl), triazolyl (e.g. triazol-1-yl), tetrazolyl (e.g. tetrazol-1-yl), oxazolyl, oxadiazolyl, thiazolyl, thiadiazolyl, thiophenyl, furanyl (e.g. furan-2-yl), pyridinyl, pyridazinyl, pyrimidinyl, pyrazinyl, triazinyl, indolyl (e.g. indol-1-yl), benzimidazolyl, benzofuranyl, benzothiophenyl, azaindolyl and azabenzofuranyl (particularly pyrrolyl (e.g. pyrol-1-yl), pyrazolyl (e.g. pyrazol-1-yl) and furanyl (e.g. furan-2-yl)), each of which heteroaryl groups is optionally substituted by one or more (e.g. one) G² groups, wherein G¹ and G² are as defined herein.

Other compounds of the invention that may be mentioned include those in which R³ represents phenyl optionally substituted by one or more (e.g. one) G¹ groups; a 5-9-membered heteroaryl group selected from pyrrolyl (e.g. pyrol-1-yl), pyrazolyl (e.g. pyrazol-1-yl), imidazolyl (e.g. imidazol-1-yl), triazolyl (e.g. triazol-1-yl), tetrazolyl (e.g tetrazol-1-yl), thiophenyl, furanyl (e.g. furan-2-yl) oxazolyl, oxadiazolyl, thiazolyl, thiadiazolyl, indolyl, benzimidazolyl, pyridinyl, pyridazinyl, pyrimidinyl, pyrazinyl, triazinyl, benzofuranyl, benzothiophenyl, azaindolyl and azabenzofuranyl (particularly pyrrolyl (e.g. pyrol-1-yl), pyrazolyl (e.g. pyrazol-1-yl) and furanyl (e.g. furan-2-yl)) each of which heteroaryl groups is optionally substituted by one or more (e.g. one) G² groups; each of which phenyl and heteroaryl groups is optionally linked to the Q¹ to Q³-containing ring via an ethynylene group; a 4-6-membered heterocyclyl group selected from azetidinyl (e.g. azetidin-1-yl) piperidinyl (e.g. piperidin-1-yl), piperizinyl (e.g. piperizin-1-yl), morpholinyl (e.g. morpholin-4-yl), pyrrolidinyl (e.g. pyrollidin-1-yl), pyrazolidinyl (e.g. pyrazolidin-1-yl), imidazolidinyl (e.g. imidazolidin-1-yl), imidazolinyl (e.g. imidazolin-1-yl or imidazolin-2-yl) (particularly piperidinyl (e.g. piperidin-1-yl)), each of which 4-6-membered heterocyclyl groups is optionally substituted by one or more (e.g. one) G³ groups; or C₁₋₄ alkyl (e.g. methyl, ethyl, propyl, butyl, 2-butyl, tent-butyl or, particularly, iso-propyl) optionally substituted by one or more (e.g. one) G⁴ groups, wherein the G¹, G², G³ and G⁴ groups are as defined herein.

Other compounds of the invention that may be mentioned include those in which R³ represents phenyl optionally substituted by one or more (e.g. one) G¹ groups; or a 5-9-membered heteroaryl group selected from pyrrolyl (e.g. pyrol-1-yl), pyrazolyl (e.g. pyrazol-1-yl), imidazolyl (e.g. imidazol-1-yl), triazolyl (e.g. triazol-1-yl), tetrazolyl (e.g tetrazol-1-yl), thiophenyl, furanyl (e.g. furan-2-yl) and indolyl (e.g. indol-1-yl), each of which heteroaryl groups is optionally substituted by one or more G² groups; wherein the G¹ and G² groups are as defined herein.

Other compounds of the invention that may be mentioned include those in which R³ represents phenyl optionally substituted by one or more (e.g. one) G¹ groups; a 5-9-membered heteroaryl group selected from pyrrolyl (e.g. pyrol-1-yl), pyrazolyl (e.g. pyrazol-1-yl), imidazolyl (e.g. imidazol-1-yl), triazolyl (e.g. triazol-1-yl), tetrazolyl (e.g. tetrazol-1-yl) and furanyl (e.g. furan-2-yl) (particularly pyrrolyl (e.g. pyrol-1-yl), pyrazolyl (e.g. pyrazol-1-yl) and furanyl (e.g. furan-2-yl)) each of which heteroaryl groups is optionally substituted by one or more (e.g. one) G² groups; a 4-6-membered heterocyclyl group selected from azetidinyl (e.g. azetidin-1-yl), piperidinyl (e.g. piperidin-1-yl), piperizinyl (e.g. piperizin-1-yl), morpholinyl (e.g. morpholin-4-yl), pyrrolidinyl (e.g. pyrolidin-1-yl), pyrazolidinyl (e.g. pyrazolidin-1-yl), imidazolidinyl (e.g. imidazolidin-1-yl), imidazolinyl (e.g. imidazolin-1-yl or imidazolin-2-yl) (particularly piperidinyl (e.g. piperidin-1-yl)), each of which 4-6-membered heterocyclyl groups is optionally substituted by one or more (e.g. one) G³ groups; C₁₋₄ alkyl (e.g. methyl, ethyl, propyl, butyl, 2-butyl, tent-butyl or, particularly, iso-propyl) or C₁₋₄ alkoxy (e.g. —Omethyl, —Oethyl, —Opropyl, —Obutyl, -02-butyl, —Otert-butyl or, particularly, —Oiso-propyl), or —N(C₁₋₄ alkyl)(C₁₋₄ alkyl) (e.g. —N(methyl)₂, —N(ethyl)₂, —N(methyl)(ethyl), —N(propyl)₂, —N(butyl)₂ (particularly —N(methyl)₂)) which latter three groups are optionally substituted by one or more (e.g. one) G⁴ groups, wherein the G¹, G², G³ and G⁴ groups are as defined herein.

Other compounds of the invention that may be mentioned include those in which R³ represents phenyl optionally substituted by one or more (e.g. one) G¹ groups; a 5-membered heteroaryl group selected from pyrrolyl (e.g. pyrol-1-yl), pyrazolyl (e.g. pyrazol-1-yl), imidazolyl (e.g. imidazol-1-yl), triazolyl (e.g. triazol-1-yl) tetrazolyl (e.g. tetrazol-1-yl), and furanyl (e.g. furan-2-yl) (particularly pyrrolyl (e.g. pyrol-1-yl), pyrazolyl (e.g. pyrazol-1-yl) and furanyl (e.g. furan-2-yl)) each of which heteroaryl groups is optionally substituted by one or more (e.g. one) G² groups; a 4-6-membered heterocyclyl group selected from azetidinyl (e.g. azetidin-1-yl) piperidinyl (e.g. piperidin-1-yl), piperizinyl (e.g. piperizin-1-yl), morpholinyl (e.g. morpholin-4-yl), pyrrolidinyl (e.g. pyrollidin-1-yl), pyrazolidinyl (e.g. pyrazolidin-1-yl), imidazolidinyl (e.g. imidazolidin-1-yl), imidazolinyl (e.g. imidazolin-1-yl or imidazolin-2-yl) (particularly piperidinyl (e.g. piperidin-1-yl)), each of which 4-6-membered heterocyclyl groups is optionally substituted by one or more (e.g. one) G³ groups; or C₁₋₄ alkyl (e.g. methyl, ethyl, propyl, butyl, 2-butyl, tent-butyl or, particularly, iso-propyl) optionally substituted by one or more (e.g. one) G⁴ groups, wherein the G¹, G², G³ and G⁴ groups are as defined herein.

Other compounds of the invention that may be mentioned include those in which R³ represents phenyl optionally substituted by one or more (e.g. one) G¹ groups; a 5-membered heteroaryl group selected from pyrrolyl (e.g. pyrol-1-yl), pyrazolyl (e.g. pyrazol-1-yl), imidazolyl (e.g. imidazol-1-yl), triazolyl (e.g. triazol-1-yl) tetrazolyl (e.g. tetrazol-1-yl), and furanyl (e.g. furan-2-yl) (particularly pyrrolyl (e.g. pyrol-1-yl), pyrazolyl (e.g. pyrazol-1-yl) and furanyl (e.g. furan-2-yl)) each of which heteroaryl groups is optionally substituted by one or more (e.g. one) G² groups, wherein the G¹ and G² groups are as defined herein.

Other compounds of the invention that may be mentioned include those in which R³ represents phenyl optionally substituted by one or more (e.g. one) G¹ groups; a 5-9-membered heteroaryl group selected from pyrrolyl (e.g. pyrol-1-yl), pyrazolyl (e.g. pyrazol-1-yl), and furanyl (e.g. furan-2-yl) and indolyl (e.g. indol-1-yl), each of which heteroaryl groups is optionally substituted by one or more (e.g. one) G² groups, wherein the G¹ and G² groups are as defined herein.

Other compounds of the invention that may be mentioned include those in which R³ represents phenyl optionally substituted by one or more (e.g. one) G¹ groups; a 5-9-membered heteroaryl group selected from pyrrolyl (e.g. pyrrol-1-yl), pyrazolyl (e.g. pyrazol-1-yl), and furanyl (e.g. furan-2-yl) and indolyl (e.g. indol-1-yl), each of which heteroaryl groups is optionally substituted by one or more (e.g. one) G² groups, wherein the G¹ and G² groups are as defined herein.

Other compounds of the invention that may be mentioned include those in which R³ represents a 5-9-membered heteroaryl group selected from pyrrolyl (e.g. pyrrol-1-yl), pyrazolyl (e.g. pyrazol-1-yl), and furanyl (e.g. furan-2-yl) and indolyl (e.g. indol-1-yl), each of which heteroaryl groups is optionally substituted by one or more (e.g. one) G² groups, wherein the G² groups are as defined herein.

Other compounds of the invention that may be mentioned include those in which R³ represents a 5-9-membered heteroaryl group selected from pyrrolyl (e.g. pyrol-1-yl), pyrazolyl (e.g. pyrazol-1-yl), and indolyl (e.g. indol-1-yl), each of which heteroaryl groups is optionally substituted by one or more (e.g. one) G² groups, wherein the G² groups are as defined herein.

Other compounds of the invention that may be mentioned include those in which R³ represents a 5-membered heteroaryl group selected from pyrrolyl (e.g. pyrol-1-yl), pyrazolyl (e.g. pyrazol-1-yl), and furanyl (e.g. furan-2-yl), each of which heteroaryl groups is optionally substituted by one or more (e.g. one) G² groups, wherein the G² groups are as defined herein.

Other compounds of the invention that may be mentioned include those in which R³ represents a 5-membered heteroaryl group selected from pyrrolyl (e.g. pyrrol-1-yl), and pyrazolyl (e.g. pyrazol-1-yl), each of which heteroaryl groups is optionally substituted by one or more (e.g. one) G² groups, wherein the G² groups are as defined herein.

R³ represents a 5-9-membered heteroaryl group selected from pyrrolyl (e.g. pyrrol-1-yl), pyrazolyl (e.g. pyrazol-1-yl), and furanyl (e.g. furan-2-yl) and indolyl (e.g. indol-1-yl) (preferably pyrrolyl (e.g. pyrrol-1-yl) pyrazolyl (e.g. pyrazol-1-yl)) or indolyl (e.g. indol-1-yl), more preferably pyrrolyl (e.g. pyrrol-1-yl)), each of which heteroaryl groups is optionally substituted by one or more (e.g. one) G² groups.

Other compounds of the invention that may be mentioned include thoase in which R³ represents indolyl (e.g. indol-1-yl) or, preferably pyrrolyl (e.g. pyrrol-1-yl)

Compounds of the invention that may be mentioned include those in which G¹ represents halogen; hydroxy; cyano; or C₁₋₄ alkyl, C₁₋₄ alkoxy or -N (C₁₋₄ alkyl)(C₁₋₄ alkyl), wherein each of the latter three groups is optionally substituted by one or more fluoro groups.

Compounds of the invention that may be mentioned include those in which G¹ represents fluoro; chloro; bromo; C₁₋₂ alkyl, C₁₋₂ alkoxy or —N(C₁₋₂ alkyl)(C₁₋₂ alkyl), wherein each of the latter three groups is optionally substituted by one or more (e.g. one) fluoro groups.

Further compounds of the invention that may be mentioned include those in which G¹ represents fluoro, chloro, methyl, ethyl, —CF₃, —CHF₂, —CH₂F, —OMe, —OEt, —OCF₃.

Further compounds of the invention that may be mentioned include those in which G¹ represents C₁₋₂ alkyl optionally substituted by one or more fluoro groups (for example methyl, ethyl, —CH₂F, —CHF₂, —CF₃, —CH₂CF₃, e.g. methyl).

Compounds of the invention that may be mentioned include those in which G² represents halogen; hydroxy; cyano; or C₁₋₄ alkyl, C₁₋₄ alkoxy or -N (C₁₋₄ alkyl)(C₁₋₄ alkyl), wherein each of the latter three groups is optionally substituted by one or more fluoro groups; or phenyl or a 5-9-membered heteroaryl group, each of which two groups is optionally substituted by one or more groups selected from halo, —CN, C₁₋₂ alkyl or C₁₋₂ alkoxy.

Compounds of the invention that may be mentioned include those in which G² represents fluoro; chloro; bromo; C₁₋₂ alkyl, C₁₋₂ alkoxy or —N(C₁₋₂ alkyl)(C₁₋₂ alkyl), wherein each of the latter three groups is optionally substituted by one or more (e.g. one) fluoro groups; or phenyl or a 5-6-membered heteroaryl group, each of which two groups is optionally substituted by one or more (e.g. one) groups selected from fluoro, chloro, methyl or methoxy groups (e.g unsubstituted).

Compounds of the invention that may be mentioned include those in which G² represents fluoro; chloro; bromo; C₁₋₂ alkyl, C₁₋₂ alkoxy or —N(C₁₋₂ alkyl)(C₁₋₂ alkyl), wherein each of the latter three groups is optionally substituted by one or more (e.g. one) fluoro groups.

Further compounds of the invention that may be mentioned include those in which G² represents fluoro, chloro, methyl, ethyl, —CF₃, —CHF₂, —CH₂F, —OMe, —OEt, —OCF₃ or —NMe₂.

Further compounds of the invention that may be mentioned include those in which G² represents C₁₋₂ alkyl optionally substituted by one or more fluoro groups (for example methyl, ethyl, —CH₂F, —CHF₂, —CF₃, —CH₂CF₃, e.g. methyl).

Compounds of the invention that may be mentioned include those in which G³ represents fluoro; chloro; bromo; C₁₋₂ alkyl or C₁₋₂ alkoxy, wherein each of the latter two groups is optionally substituted by one or more (e.g. one) fluoro groups; or phenyl or a 5-6-membered heteroaryl group, each of which two groups is optionally substituted by one or more (e.g. one) groups selected from fluoro, chloro, methyl or methoxy groups (e.g unsubstituted).

Compounds of the invention that may be mentioned include those in which G³ represents fluoro; chloro; bromo; C₁₋₂ alkyl or C₁₋₂ alkoxy, wherein each of the latter two groups is optionally substituted by one or more (e.g. one) fluoro groups.

Further compounds of the invention that may be mentioned include those in which G³ represents fluoro, chloro, methyl, ethyl, —CF₃, —CHF₂, —CH₂F, —OMe, —OEt, or —OCF₃.

Further compounds of the invention that may be mentioned include those in which G³ represents or C₁₋₂ alkyl optionally substituted by one or more fluoro groups (for example methyl, ethyl, —CH₂F, —CHF₂, —CF₃, —CH₂CF₃, e.g. methyl).

Compounds of the invention that may be mentioned include those in which G⁴ represents fluoro, chloro, bromo or a C₁₋₂ alkoxy group, wherein the C₁₋₂ alkoxy group is optionally substituted by one or more fluoro groups (e.g. —OMe, —OEt, —OCH₂F, —OCHF₂, —OCF₃, —OCH₂CF₃).

Further compounds of the invention that may be mentioned include those in which G⁴ represents fluoro, chloro, —OMe, —OEt, —OCH₂F, —OCHF₂, —OCF₃, —OCH₂CF₃.

Further compounds of the invention that may be mentioned include those in which R³ represents:

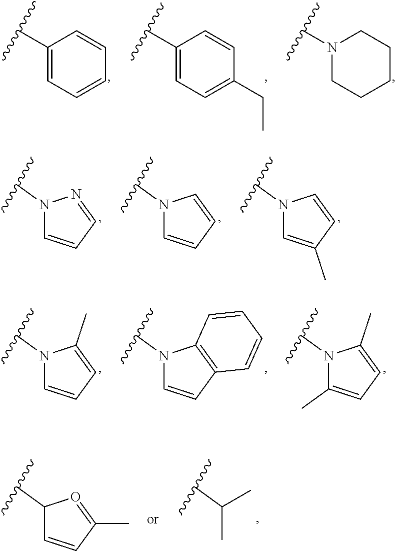

wherein [symbol] indicates the point of attachment to the phenyl ring.

Further compounds of the invention that may be mentioned include those in which R³ represents

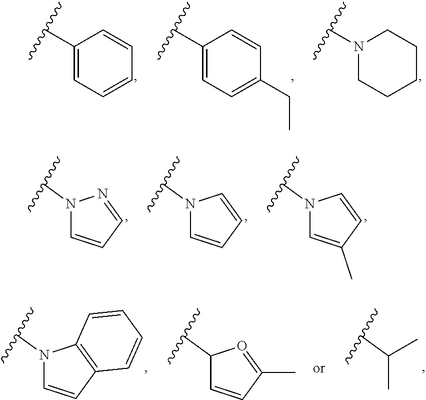

wherein [symbol] indicates the point of attachment to the phenyl ring.

Further compounds of the invention that may be mentioned include those in which R³ represents:

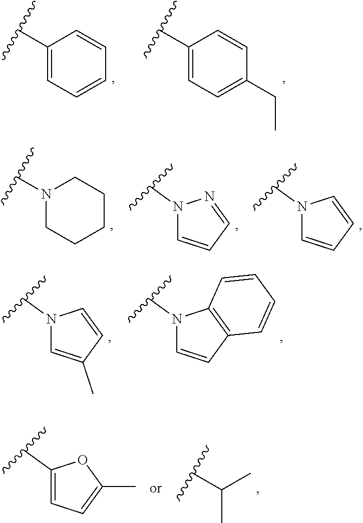

wherein [symbol] indicates the point of attachment to the phenyl ring.

Further compounds of the invention that may be mentioned include those in which R³ represents:

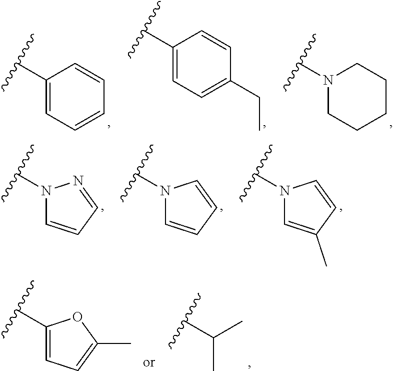

wherein [symbol] indicates the point of attachment to the phenyl ring.

Furter compounds of the invention that may be mentioned include those in which R³ represents:

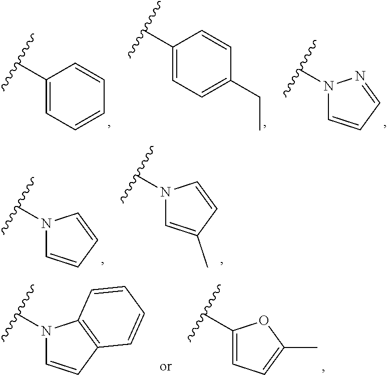

wherein [symbol] indicates the point of attachment to the phenyl ring.

Further compounds of the invention that may be mentioned include those in which R³ represents

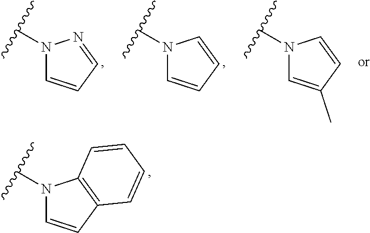

wherein [symbol] indicates the point of attachment to the phenyl ring.

Compounds of the invention that may be mentioned include those in which R³ represents phenyl optionally substituted by one or more (e.g. one) G¹ group; a 5-9-membered heteroaryl group (for example, a 5-6-membered heteroaryl group, e.g. a 5-membered heteroaryl group) optionally substituted by one or more (e.g. one) G² groups; a 4-7-membered heterocyclyl group (for example a 5-6-membered heterocyclyl group, e.g. a 6-membered heterocyclyl group) optionally substituted by one or more (e.g. one) G³ groups, wherein the G¹, G² and G³ groups are as defined herein.

Compounds of the invention that may be mentioned include those in which R³ represents phenyl optionally substituted by one or more (e.g. one) G¹ groups; a 5-9-membered heteroaryl group (e.g. for example, a 5-6-membered heteroaryl group, e.g. a 5-membered heteroaryl group) optionally substituted by one or more (e.g. one) G² groups; wherein the G¹ and G² groups are as defined herein.

Further compounds of the invention that may be mentioned include those in which R³ represents phenyl optionally substituted by one methyl or ethyl group (e.g. 4-methylphenyl or 4-ethylphenyl); pyrrolyl (e.g. pyrrol-1-yl) optionally substituted by one to four methyl groups (e.g. one or two); pyrazolyl (e.g. pyrazol-1-yl) optionally substituted by one to three methyl groups (e.g. one or two); or furanyl optionally substituted by one to three methyl groups (e.g. one or two).

Further compounds of the invention that may be mentioned include those in which R³ represents phenyl optionally substituted by one methyl or ethyl group (e.g. 4-methylphenyl or 4-ethylphenyl); pyrrolyl (e.g. pyrrol-1-yl) optionally substituted by one to four methyl groups (e.g. one or two); or piperidinyl (e.g. piperidin-1-yl).

Compounds of the invention that may be mentioned include those in which one of Q¹, Q² and Q³ represents —N— and the others represent —C(R⁴)—.

Compounds of the invention that may be mentioned include those in which Q¹, Q² and Q³ each represent —C(R⁴)—.

Compounds of the invention that may be mentioned include those in which R⁴ represents H, halogen, C₁₋₄ alkyl or C₁₋₄ alkoxy, which latter two groups are optionally substituted by one or more fluoro groups.

Compounds of the invention that may be mentioned include those in which each R⁴ group independently represents H, chloro, bromo, C₁₋₂ alkyl (e.g. methyl) or C₁₋₂ alkoxy (e.g methoxy), which latter two groups are optionally substituted by one or more fluoro groups (e.g. methyl, ethyl, —CF₃, —CHF₂, CH₂F, —CH₂CF₃ —Omethyl, —Oethyl, —OCF₃, —OCHF₂, —OCH₂F or —OCH₂CF₃ (particularly methyl or —Omethyl)).

Further compounds of the invention that may be mentioned include those in which one R⁴ group represents chloro, bromo, C₁₋₂ alkyl or C₁₋₂ alkoxy, which latter two groups are optionally substituted by one or more fluoro groups (e.g. methyl, ethyl, —CF₃, —CHF₂, CH₂F, —CH₂CF₃ —Omethyl, —Oethyl, —OCF₃, —OCHF₂, —OCH₂F or —OCH₂CF₃ (particularly methyl or —Omethyl)). and the remaining R⁴ groups represent H.

Further compounds of the invention include those in which each R⁴ group represents H (i.e. Q¹, Q² and Cr independently represent —N— or —CH—, wherein a maximum of two (e.g. one) of Q¹ to Cr may represent —N—).

Further compounds of the invention that may be mentioned include those in which Q¹, Q² and Q³ each represent —CH—.

Compounds of the invention that may be mentioned include those in which R^{a1}, R^{a2}, R^{a3}, R^{a4}, R^{a5}, R^{a6}, R^{a7}, R^{a8}, R^{a9} and R^{a10} each independently represent H or C₁₋₂ alkyl, which C₁₋₂ alkyl groups are optionally substituted by one or more fluoro groups; or

R^{a1} and R^{a2}, R^{a3} and R^{a4}, R^{a5} and R^{a6}, R^{a7} and R^{a8}, R^{a9} and R^{a10} may be independently be joined together to form, together with the atom to which they are attached, a 4- to 7-membered heterocyclyl ring, which heterocyclyl ring optionally contains one further heteroatom selected from N, O and S (e.g. N or O).

Further compounds of the invention that may be mentioned include those in which R^{a1}, R^{a2}, R^{a3}, R^{a4}, R^{a5}, R^{a6}, R^{a7}, R^{a8}, R^{a9} and R^{a10} each independently represent H or C₁₋₂ alkyl, which C₁₋₂ alkyl groups are optionally substituted by one or more fluoro groups.

Particular compounds of the invention that may be mentioned include those in which:

R¹ represents methyl; phenyl optionally substituted by one or more (e.g. one) groups selected from fluoro, chloro, methyl, ethyl, methoxy, ethoxy, —CF₃, —OCF₃ or methylenedioxy; thiophenyl optionally substituted by one or more (e.g. one) methyl group (e.g. thiophen-2-yl, thiophen-3-yl or 5-methylthiophen-2-yl); pyrazolyl optionally substituted by one or more (e.g. one) methyl or phenyl groups (e.g. pyrazol-3-yl, pyrazol-4-yl, pyrazol-5-yl or, particularly, 1-phenylpyrazol-4-yl); thiazolyl (e.g. thiazol-2-yl, thiazol-4-yl or thiazol-5-yl); pyridinyl (e.g. pyridine-2-yl, pyridine-3-yl or pyridin-4-yl); benzofuranyl (e.g. benzofuran-5-yl; benzofuran-4-yl); or indolyl (e.g. indol-5-yl or indol-4-yl);

R² represents chloro, bromo, C₁₋₄ alkyl or C₁₋₄ alkoxy, which latter two groups are optionally substituted by one or more fluoro, ═O, hydroxy, —NMe₂ or methoxy groups;

R³ represents phenyl optionally substituted by one or more (e.g. one) G¹ group; a 5-9-membered heteroaryl group (for example, a 5-6-membered heteroaryl group, e.g. a 5-membered heteroaryl group) optionally substituted by one or more (e.g. one) G² groups; a 4-7-membered heterocyclyl group (for example a 5-6-membered heterocyclyl group, e.g. a 6-membered heterocyclyl group) optionally substituted by one or more (e.g. one) G³ groups, wherein the G¹, G² and G³ groups are as defined herein.

Q¹, Q² and Q³ each represent —C(R⁴)-;

one R⁴ group represents chloro, bromo, C₁₋₂ alkyl or C₁₋₂ alkoxy, which latter two groups are optionally substituted by one or more fluoro groups (e.g. methyl, ethyl, —CF₃, —CHF₂, CH₂F, —CH₂CF₃ —Omethyl, —Oethyl, —OCF₃, —OCHF₂, —OCH₂F or —OCH₂CF₃ (particularly methyl or —Omethyl)). and the remaining R⁴ groups represent H; or all R⁴ groups represent H (i.e. Q¹, Q² and Q³ each represent —CH—).

Further particular compounds of the invention that may be mentioned include those in which:

R¹ represents methyl; or phenyl optionally substituted by one group selected from methyl, methoxy, chloro, fluoro, —OCF₃ and methylenedioxy;

R² represents methyl, ethyl, or —CF₃;

R³ represents phenyl optionally substituted by one or more (e.g. one) G¹ groups; a 5-9-membered heteroaryl group (e.g. for example, a 5-6-membered heteroaryl group, e.g. a 5-membered heteroaryl group) optionally substituted by one or more (e.g. one) G² groups;

G¹ represents fluoro; chloro; bromo; C₁₋₂ alkyl, C₁₋₂ alkoxy or —N(C₁₋₂ alkyl)(C₁₋₂ alkyl), wherein each of the latter three groups is optionally substituted by one or more (e.g. one) fluoro groups;

G² represents fluoro; chloro; bromo; C₁₋₂ alkyl, C₁₋₂ alkoxy or —N(₁₋₂ alkyl)(C₁₋₂ alkyl), wherein each of the latter three groups is optionally substituted by one or more (e.g. one) fluoro groups.

Q¹, Q² and Q³ each represent —C(R⁴)—;

one R⁴ group represents chloro, bromo, C₁₋₂ alkyl or C₁₋₂ alkoxy, which latter two groups are optionally substituted by one or more fluoro groups (e.g. methyl, ethyl, —CF₃, —CHF₂,

CH₂F, —CH₂CF₃ —Omethyl, —Oethyl, —OCF₃, —OCHF₂, —OCH₂F or —OCH₂CF₃ (particularly methyl or —Omethyl)). and the remaining R⁴ groups represent H; or

all R⁴ groups represent H (i.e. Q¹, Q² and Q³ each represent —CH—).

Further particular compounds of the invention that may be mentioned include those in which

R¹ represents methyl; or, preferably, phenyl optionally substituted by one group selected from methyl, methoxy, chloro, fluoro, —OCF₃ and methylenedioxy, preferably R¹ represents phenyl or tolyl (o-tolyl, p-tolyl or, preferably, m-tolyl);

R² represents C₁₋₂ alkyl optionally substituted by one or more fluoro groups;

R³ represents a 5-9-membered heteroaryl group selected from pyrrolyl (e.g. pyrrol-1-yl), pyrazolyl (e.g. pyrazol-1-yl), and furanyl (e.g. furan-2-yl) and indolyl (e.g. indol-1-yl) (preferably pyrrolyl (e.g. pyrrol-1-yl) pyrazolyl (e.g. pyrazol-1-yl)) or indolyl (e.g. indol-1-yl), more preferably pyrrolyl (e.g. pyrrol-1-yl)), each of which heteroaryl groups is optionally substituted by one or more (e.g. one) G² groups

G² represents fluoro, chloro, methyl, ethyl, —CF₃, —CHF₂, —CH₂F, —OMe, —OEt, —OCF₃ or —NMe₂; preferably G² represents fluoro, ethyl or methyl (e.g. methyl);

Q¹, Q² and Q³ each represent —C(R⁴)—;

one R⁴ group represents chloro, bromo, C₁₋₂ alkyl or C₁₋₂ alkoxy, which latter two groups are optionally substituted by one or more fluoro groups (e.g. methyl, ethyl, —CF₃, —CHF₂, CH₂F, —CH₂CF₃ —Omethyl, —Oethyl, —OCF₃, —OCHF₂, —OCH₂F or —OCH₂CF₃ (particularly methyl or —Omethyl)) and the remaining R⁴ groups represent H; or, preferably,

all R⁴ groups represent H (i.e. Q¹, Q² and Q³ each represent —CH—).

Particular compounds of the invention that may be mentioned include those compounds described in the examples provided herein, and pharmaceutically-acceptable salts thereof. For the avoidance of doubt, where such compounds of the invention include compounds in particular salt form, compounds of the invention include those compounds in non-salt form and in the form of any pharmaceutically-acceptable salt thereof (which may include the salt form present in such examples).

Medical Uses

As indicated herein, the compounds of the invention, and therefore compositions and kits comprising the same, are useful as pharmaceuticals.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the active compounds to which they are metabolised) may therefore be described as “prodrugs” of compounds of the invention.

As used herein, references to prodrugs will include compounds that form a compound of the invention, in an experimentally-detectable amount, within a predetermined time, following enteral (e.g. oral) or parenteral administration. All prodrugs of the compounds of the invention are included within the scope of the invention.

Furthermore, certain compounds of the invention may possess no or minimal pharmacological activity as such, but may be administered parenterally or orally, and thereafter be metabolised in the body to form compounds of the invention that possess pharmacological activity as such. Such compounds (which also includes compounds that may possess some pharmacological activity, but that activity is appreciably lower than that of the active compounds of the invention to which they are metabolised), may also be described as “prodrugs”.

For the avoidance of doubt, compounds of the invention are therefore useful because they possess pharmacological activity, and/or are metabolised in the body following oral or parenteral administration to form compounds that possess pharmacological activity.

As described herein, compounds of the invention may be particularly useful in the treatment of diseases characterised by impaired signalling of neurotrophins and/or other trophic factors. Due to their mode of action, compounds of the invention may have particular utility in the treatment of such diseases in patients with the Val66Met mutation in the BDNF gene.

The compounds of the invention may also have particular utility in the treatment of diseases characterised by impaired signalling of neurotrophins and/or other trophic factors in patients having other genetic variations, including deletions, that directly or indirectly affect the BDNF gene. For example, the compounds of the invention may have particular utility in treating diseases in patients having the rs12291063 minor C allele, which is known to be associated with lower BDNF expression, and/or in patients having the deletions associated with WAGR syndrome, such as the deletions in chromosome 11.

Accordingly, in particular embodiments of the invention, there is provided the compounds of the invention for use in the treatment of the diseases described herein in a patient having the Val66Met mutation in the BDNF gene, and/or in a patient having the rs12291063 minor C allele, and/or in a patient having the genetic deletions associated with WAGR syndrome.

The skilled person will understand that trophic factors refer to a class of molecules that promote the growth and maintenance of cellular tissues. Neurotrophins may be understood to refer to a class of molecules associated with promoting the growth and survival of neurons, which are also referred to as neurotrophic factors. Examples of neurotrophins include NGF, BDNF, NT3 and NT4/5. Other trophic factors include insulin-like growth factor (IGF-1), fibroblast growth factors (FGFs), hepatocyte growth factor (HGF) and glial cell line-derived neurotrophic factors such as glial cell-derived neurotrophic factor (GDNF), Neurturin (NRTN), artemin (ARTN) and persephin (PSPN).

As used herein, the phrase diseases characterised by impaired signalling of neurotrophins and other trophic factors may be understood to indicate diseases and disorders that involve reduced signalling of trophic factors, such as those listed above. Such disorders may be treated through the positive modulation of neurotrophin receptors, such as TrKA, TrKB and TrkC and/or their signalling, and receptor tyrosine kinases such as FGFR1 and IGF1R and/or their signalling and/or the positive modulation of other trophic factor receptors.

The Val66Met mutation in the BDNF gene refers to a common single-nucleotide polymorphism in the brain-derived neurotrophic factor (BDNF) gene, resulting in a methionine (Met) substitution for valine (Val) at codon 66 (Val66Met).

The skilled person will understand that references to the treatment of a particular condition (or, similarly, to treating that condition) will take their normal meanings in the field of medicine. In particular, the terms may refer to achieving a reduction in the severity and/or frequency of occurrence of one or more clinical symptom associated with the condition, as adjudged by a physician attending a patient having or being susceptible to such symptoms.

For example, in the case of Alzheimer's disease, the term may refer to achieving an improvement in cognition in the patient being treated.

As used herein, the term prevention (and, similarly, preventing) will include references to the prophylaxis of the disease or disorder (and vice versa). As such, references to prevention may also be references to prophylaxis, and vice versa. In particular, such terms may refer to achieving a reduction (for example, at least a 10% reduction, such as at least a 20%, 30% or 40% reduction, e.g. at least a 50% reduction) in the likelihood of the patient (or healthy subject) developing the condition (which may be understood as meaning that the condition of the patient changes such that patient is diagnosed by a physician as having, e.g. requiring treatment for, the relevant disease or disorder).

As used herein, references to a patient (or to patients) will refer to a living subject being treated, including mammalian (e.g. human) patients.

For the avoidance of doubt, the skilled person will understand that such treatment or prevention will be performed in a patient (or subject) in need thereof. The need of a patient (or subject) for such treatment or prevention may be assessed by those skilled the art using routine techniques.

As used herein, the terms disease and disorder (and, similarly, the terms condition, illness, medical problem, and the like) may be used interchangeably.

Compounds of the invention are modulators of neurotrophin receptors, such as TrkA, TrkB, TrkC and/or their signalling and receptor tyrosine kinases, such as FGFR1 and IGF1R and/or their signalling. The compounds are believed to have an improved potency for the modulation of neurotrophin receptors, such as TrkA, TrkB, TrkC and/or their signalling and receptor tyrosine kinases, such as FGFR1 and IGF1R and/or their signalling. It is believed that the compounds of the invention would have a reduced potential for side effects associated with conventional agonists for TrkA and TrkB.

Another indication includes setting in which there is a goal for enhancing plasticity of the nervous system, such as during rehabilitation or acquisition of a new learned physical or intellectual skill. Moreover, it also includes facilitation of neuronal or non-neuronal or stem cell survival or promoting neural function by treating a neural or non-neuronal or stem cell with a compound of the invention having the ability to have a positive modulatory effect, either directly or indirectly, on the signalling mediated by the TrkA, TrkB and TrkC receptors, optionally in combination with a modulatory effect, either directly or indirectly, on on the signalling mediated by receptor tyrosine kinases such as IGF1R and/or FGFR1 receptor.

The invention relates to the compounds of the invention and pharmaceutically acceptable salts thereof, as defined above, for use in medicine (e.g. human medicine). Without being bound to theory regarding the mode of action of the compounds defined above, it is believed that the compounds can be used for treatment and/or prevention of the diseases mentioned herein.

In particular embodiments, the diseases that may be treated by compounds of the invention include Alzheimer's disease, depression, Parkinson's disease, other Parkinsonian disorders and/or other tauopathies, Lewy body dementia, multiple sclerosis, Huntington's disease, mild cognitive impairment, brain injuries (including traumatic brain injuries), stroke, other dementia disorders, motorneurone diseases, Pick disease, spinal chord injury, hypoxic ischemia injury, cognitive dysfunction, coronary artery disease, obesity, metabolic syndrome, diabetes, Charcot-Marie-Tooth disease, diabetic neuropathy (including complications thereof such as osteoporosis, painful connective tissue disorders and tendon ruptures), tissue regeneration, motor function, nerve injury, hearing loss, blindness, posterior eye diseases, dry eye disease, neurotrophic keratitis, glaucoma, high intraocular pressure (IOP), retinitis pigmentosa, post-traumatic stress disorders, WAGR syndrome, diseases of the olfactory tract, olfactory decline, olfactory dysfunction, anxiety, fragile X syndrome, congenital central hypoventilation syndrome, obsessive-compulsive disorder, generalized anxiety disorder, eating disorders, bipolar disorder, chronic fatigue syndrome, neuromyelitis optica, Rett syndrome, Friedrich's ataxia, obstructive sleep apnea-hypopnea syndrome and constipation (including, particularly, constipation in Parkinson's disease, slow-transit constipation and opioid-induced constipation).

In particular embodiments, the diseases that may be treated by compounds of the invention include Alzheimer's disease, depression, Parkinson's disease, other

Parkinsonian disorders and/or other tauopathies, Lewy body dementia, multiple sclerosis, Huntington's disease, mild cognitive impairment, brain injuries (including traumatic brain injuries), stroke, other dementia disorders, motorneurone diseases, Pick disease, spinal chord injury, hypoxic ischemia injury, cognitive dysfunction, coronary artery disease, obesity, metabolic syndrome, diabetes, Charcot-Marie-Tooth disease, diabetic neuropathy (including complications thereof such as osteoporosis (diabetes-induced osteoporosis), painful connective tissue disorders and tendon ruptures), tissue regeneration, motor function, nerve injury, hearing loss (including genetic or acquired hearing loss), blindness, posterior eye diseases, anterior eye diseases, dry eye disease, neurotrophic keratitis, glaucoma, high intraocular pressure (IOP), retinitis pigmentosa, post-traumatic stress disorders, WAGR syndrome, Prader-Willi syndrome, diseases of the olfactory tract, olfactory decline, olfactory dysfunction, anxiety, fragile X syndrome, congenital central hypoventilation syndrome, obsessive-compulsive disorder, generalized anxiety disorder, eating disorders, bipolar disorder, chronic fatigue syndrome, neuromyelitis optica, Rett syndrome, Friedrich's ataxia, obstructive sleep apnea-hypopnea syndrome and constipation (including, particularly, constipation in Parkinson's disease, slow-transit constipation and opioid-induced constipation).

In particular embodiments, the diseases that may be treated by compounds of the invention include Alzheimer's disease, depression, Parkinson's disease, other Parkinsonian disorders and/or other tauopathies, Lewy body dementia, multiple sclerosis, Huntington's disease, mild cognitive impairment, brain injuries (including traumatic brain injuries), stroke, other dementia disorders, motorneurone diseases, Pick disease, spinal chord injury, hypoxic ischemia injury, cognitive dysfunction, coronary artery disease, obesity, metabolic syndrome, diabetes, Charcot-Marie-Tooth disease, diabetic neuropathy (including complications thereof such as osteoporosis, painful connective tissue disorders and tendon ruptures), tissue regeneration, motor function, nerve injury, hearing loss, blindness, posterior eye diseases, dry eye disease, neurotrophic keratitis, glaucoma, high intraocular pressure (IOP), retinitis pigmentosa, post-traumatic stress disorders, WAGR syndrome, diseases of the olfactory tract, olfactory decline, olfactory dysfunction, anxiety, fragile X syndrome, congenital central hypoventilation syndrome, obsessive-compulsive disorder, generalized anxiety disorder, eating disorders, bipolar disorder, chronic fatigue syndrome, neuromyelitis optica, Rett syndrome, Friedrich's ataxia and obstructive sleep apnea-hypopnea syndrome.

As used herein, the phrase “other Parkinsonian disorders” may be understood to refer to disorders that have symptoms similar to Parkinson's disease, such as bradykinesia, tremors and postural instability. Examples of such disorders include progressive supranuclear palsy (PSP), multiple system atrophy (MSA), and corticobasal degeneration (CBD).

The phrase “other tauopathies” may be understood to refer to neurodegenerative diseases other than Alzheimer's disease that are associated with the pathological misfolding of tau protein in the brain. Examples of such disorders include primary age-related tauopathy, progressive supranuclear palsy, Pick's disease, corticobasal degeneration and post-encephalitic parkinsonism. The skilled person will understand that certain disorders such as progressive supranuclear palsy may be described as both a Parkinsonian disorder and lo a tauopathy.

The phrase “other dementia disorders” may be understood to include vascular dementia, mixed vascular dementia, incident dementia, post-operative dementia, presenile dementia, dementia associated with Parkinson's disease and dementia due to HIV infection. Progressive supranuclear palsy and corticobasal degeneration may also be classed as dementia disorders.

Motor neurone diseases include amyotrophic lateral sclerosis (ALS), hereditary spastic paraplegia (HSP), primary lateral sclerosis (PLS), progressive muscular atrophy (PMA), progressive bulbar palsy (PBP) and pseudobulbar palsy.

Cognitive dysfunction may be understood to refer to reduced cognitive abilities in a patient including reduced ability in learning, memory loss, perception, and problem solving. Cognitive dysfunction is associated with a range of conditions, such as Alzheimer's disease, Parkinson's disease, progressive supranuclear palsy, corticobasal degeneration and schizophrenia. Accordingly, in particular embodiments, the compounds of the invention may be used in the treatment of cognitive dysfunction in Alzheimer's disease, Parkinson's disease, progressive supranuclear palsy, corticobasal degeneration or schizophrenia. Cognitive dysfunction also includes post-operative cognitive dysfunction and impaired cognition associated with preterm delivery.

Similarly, in other particular embodiments, the compounds of the invention may be used in improving cognition in a patient with Alzheimer's disease, Parkinson's disease, progressive supranuclear palsy, corticobasal degeneration or schizophrenia. As used herein, the phrase “improving cognition” may be understood to indicate enhancing a patient's learning, memory, perception, and/or problem-solving ability. Improving cognition may also refer to slowing or arresting the rate of decline in cognition in a patient suffering from cognitive dysfunction (e.g. associated with the disorders listed above).

Cognitive function may be assessed using standard tests known to the person skilled in the art. Examples of such tests include the Alzheimer's Disease Assessment Scale-Cognitive subscale test (ADAS-COG) the Mini-Mental State Examination (MMSE), the Clinical Dementia Rating (CDR) the Clinical Dementia Rating-Sum of Boxes (CDR-SB), the Alzheimer's Disease Cooperative Study-Preclinical Alzheimer Cognitive Composite (ADCS-PACC) and the Repeatable Battery for the Assessment of Neuropsychological Status (RBANS) test.

As used herein, “eating disorders” may be understood to include hyperphagia, anorexia nervosa, restricting anorexia nervosa and bulimia nervosa.

In accordance with a further aspect of the invention, there is provided the compounds of the invention, or a pharmaceutically acceptable salt thereof, for use in treatment and/or prevention of one or more disease selected from the group comprising or containing Alzheimer's disease, Lewy body dementia, frontotemporal dementia, HIV dementia, Huntington's disease, amyotrophic lateral sclerosis and other motor neurone diseases, Rett syndrome, epilepsy, Parkinson's disease and other parkinsonian disorders, disorders in which enhancement of nerve regeneration is beneficial, such as demyelinating diseases including multiple sclerosis, spinal cord injury, stroke, hypoxia, ischemia, brain injury including traumatic brain injury, mild cognitive impairment, dementia disorders (including dementia of mixed vascular and degenerative origin, presenile dementia, senile dementia and dementia associated with Parkinson's disease, progressive supranuclear palsy or corticobasal degeneration) and cognitive dysfunction in schizophrenia, obesity, diabetes and metabolic syndrome, diabetic neuropathy including associated disorders such as osteoporosis, painful connective tissue disorders and tendon ruptures, Charcot Marie Tooth disease and its variants, nerve transplantation and its complications, motor neurone disease, peripheral nerve injury, genetic or acquired or traumatic hearing loss, blindness and posterior eye diseases, depression, obesity, metabolic syndrome, pain, depression, schizophrenia, anxiety and constipation, particularly, constipation in Parkinson's disease, slow-transit constipation and opioid-induced constipation.

In accordance with a further aspect of the invention, there is provided the compounds of the invention, or a pharmaceutically acceptable salt thereof, for use in treatment and/or prevention of one or more disease selected from the group comprising or containing Alzheimer's disease, Lewy body dementia, frontotemporal dementia, HIV dementia, Huntington's disease, amyotrophic lateral sclerosis and other motor neurone diseases, Rett syndrome, epilepsy, Parkinson's disease and other parkinsonian disorders, disorders in which enhancement of nerve regeneration is beneficial, such as demyelinating diseases including multiple sclerosis, spinal cord injury, stroke, hypoxia, ischemia, brain injury including traumatic brain injury, mild cognitive impairment, dementia disorders (including dementia of mixed vascular and degenerative origin, presenile dementia, senile dementia and dementia associated with Parkinson's disease, progressive supranuclear palsy or corticobasal degeneration) and cognitive dysfunction in schizophrenia, obesity, diabetes and metabolic syndrome, diabetic neuropathy including associated disorders such as osteoporosis, (diabetes-induced osteoporosis), painful connective tissue disorders and tendon ruptures, Charcot Marie Tooth disease and its variants, nerve transplantation and its complications, motor neurone disease, peripheral nerve injury, genetic or acquired or traumatic hearing loss, blindness and posterior eye diseases, anterior eye diseases, depression, obesity, metabolic syndrome, pain, depression, schizophrenia, anxiety and constipation, particularly, constipation in Parkinson's disease, slow-transit constipation and opioid-induced constipation.

In accordance with a further aspect of the invention, there is provided the compounds of the invention, or a pharmaceutically acceptable salt thereof, for use in treatment and/or prevention of one or more disease selected from the group comprising or containing Alzheimer's disease, Lewy body dementia, frontotemporal dementia, HIV dementia, Huntington's disease, amyotrophic lateral sclerosis and other motor neurone diseases, Rett syndrome, epilepsy, Parkinson's disease and other parkinsonian disorders, disorders in which enhancement of nerve regeneration is beneficial, such as demyelinating diseases including multiple sclerosis, spinal cord injury, stroke, hypoxia, ischemia, brain injury including traumatic brain injury, mild cognitive impairment, dementia disorders (including dementia of mixed vascular and degenerative origin, presenile dementia, senile dementia and dementia associated with Parkinson's disease, progressive supranuclear palsy or corticobasal degeneration) and cognitive dysfunction in schizophrenia, obesity, diabetes and metabolic syndrome, diabetic neuropathy including associated disorders such as osteoporosis, painful connective tissue disorders and tendon ruptures, Charcot Marie Tooth disease and its variants, nerve transplantation and its complications, motor neurone disease, peripheral nerve injury, genetic, acquired or traumatic hearing loss, blindness and posterior eye diseases, depression, obesity, metabolic syndrome, pain, depression, schizophrenia and anxiety.

In more particular embodiments, the disease characterised by impaired signalling of neurotrophins and/or other trophic factors is selected from the group consisting of Alzheimer's disease, Parkinson's disease, other Parkinsonian diseases, other tauopathies, Lewy body dementia, motor neurone disease, Pick disease, obesity, metabolic syndrome, diabetes, diabetic neuropathy, constipation and Rett syndrome.

In more particular embodiments, the disease characterised by impaired signalling of neurotrophins and/or other trophic factors is selected from the group consisting of Alzheimer's disease, Parkinson's disease, other Parkinsonian diseases, other tauopathies, Lewy body dementia, motor neurone disease, Pick disease, obesity, metabolic syndrome, diabetes, diabetic neuropathy and Rett syndrome.

In more particular embodiments, the disease characterised by impaired signalling of neurotrophins and/or other trophic factors is selected from the group consisting of Alzheimer's disease, Parkinson's disease, other Parkinsonian diseases, other tauopathies, Lewy body dementia, motor neurone disease, Pick disease, obesity, metabolic syndrome, diabetes and Rett syndrome. The treatment of this group of disorders may be particularly effective in patients having the Val66Met mutation in the BDNF gene.

In yet more particular embodiments, the disease characterised by impaired signalling of neurotrophins and/or other trophic factors is an eye disorder.

In yet more particular embodiments, the disease characterised by impaired signalling of neurotrophins and/or other trophic factors is an eye disorder selected from the group consisting of blindness, posterior eye diseases, anterior eye diseases, dry eye disease, neurotrophic keratitis, glaucoma, high intraocular pressure and retinitis pigmentosa. More particularly, the eye disorder is selected from the group consisting of dry eye disease, neurotrophic keratitis and glaucoma.

In yet more particular embodiments, the disease characterised by impaired signalling of neurotrophins and/or other trophic factors is selected from the group consisting of Alzheimer's disease, Parkinson's disease, Cognitive dysfunction, depression, diabetic neuropathy, constipation and Rett Syndrome.

In yet more particular embodiments, the disease characterised by impaired signalling of neurotrophins and/or other trophic factors is selected from the group consisting of Alzheimer's disease, Parkinson's disease, Cognitive dysfunction, depression, diabetic neuropathy and Rett Syndrome.

In yet more particular embodiments, the disease characterised by impaired signalling of neurotrophins and/or other trophic factors is selected from the group consisting of

Alzheimer's disease, Parkinson's disease, Cognitive dysfunction, depression and Rett Syndrome.

A further embodiment of the invention relates to a compound of the invention, or a pharmaceutically acceptable salt thereof, for use in treatment and/or prevention of

Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and/or other Parkinsonian disorders.

A further embodiment of the invention relates to a compound of the invention, or a pharmaceutically acceptable salt thereof, for use in treatment and/or prevention of Alzheimer's disease, Parkinson's disease, Cognitive dysfunction in Schizophrenia, Rett's Syndrome and/or depression.

A further embodiment of the invention relates to a compound of the invention, or a pharmaceutically acceptable salt thereof, for use in treatment and/or prevention of Alzheimer's disease.

A further embodiment of the invention relates to a compound of the invention, or a pharmaceutically acceptable salt thereof, for use in treatment and/or prevention of depression.

A further embodiment of the invention relates to a compound of the invention, or a pharmaceutically acceptable salt thereof, for use in treatment and/or prevention of a disease where enhancement of nerve regeneration is beneficial, such as demyelinating diseases.

A further embodiment of the invention relates to a compound of the invention, or a pharmaceutically acceptable salt thereof, for use in treatment and/or prevention of multiple sclerosis.

A further embodiment of the invention relates to a compound of the invention, or a pharmaceutically acceptable salt thereof, for use in treatment and/or prevention of Rett syndrome.

A further embodiment of the invention relates to a compound of the invention, or a pharmaceutically acceptable salt thereof, for use in treatment and/or prevention spinal cord injury, stroke, hypoxia, ischemia and/or brain injury including traumatic brain injury.

A further embodiment of the invention relates to a compound of the invention, or a pharmaceutically acceptable salt thereof, for use in the treatment and/or prevention of mild cognitive impairment, dementia disorders (including dementia of mixed vascular and degenerative origin, presenile dementia, senile dementia and dementia associated with Parkinson's disease, progressive supranuclear palsy, corticobasal degeneration, post-operative dementia) and/or cognitive dysfunction in schizophrenia.

A further embodiment of the invention relates to a compound of the invention, or a pharmaceutically acceptable salt thereof, for use in the treatment and/or prevention of atherosclerosis, obesity, diabetes and metabolic syndrome, diabetic neuropathy including complications thereof such as osteoporosis, painful connective tissue disorders and tendon ruptures, Charcot Marie Tooth disease and its variants, nerve transplantation and its complications, diabetes-induced osteoporosis, motor neurone disease, peripheral nerve injury, genetic or acquired or traumatic hearing loss, blindness and posterior eye diseases, depression, obesity, metabolic syndrome, WAGR syndrome, Prader Willi syndrome and/or pain.

A further embodiment of the invention relates to a compound of the invention, or a pharmaceutically acceptable salt thereof, for use in the treatment and/or prevention of atherosclerosis, obesity, diabetes and metabolic syndrome, diabetic neuropathy including complications thereof such as osteoporosis, painful connective tissue disorders and tendon ruptures, Charcot Marie Tooth disease and its variants, nerve transplantation and its complications, motor neurone disease, peripheral nerve injury, genetic or acquired or traumatic hearing loss, blindness and posterior eye diseases, depression, obesity, metabolic syndrome and/or pain.

A further embodiment of the invention relates to a compound of the invention, or a pharmaceutically acceptable salt thereof, for use in the treatment and/or prevention of depression, schizophrenia and/or anxiety.

Another embodiment relates to a use of a compound of the invention, or a pharmaceutically acceptable salt thereof, for the treatment and/or prevention of a disease in which modulators of neurotrophin receptors, such as TrkA, TrkB, TrkC and/or their signalling and receptor tyrosine kinases, such as FGFR1 and IGF1R and/or their signalling are beneficial, such as for the treatment and/or prevention of both non-neurological and neurological diseases, including one or more of the conditions mentioned hereinbefore.

The invention further relates to the use of a compound of the invention in a method of treating, preventing or reducing the risk of a disease in which modulators of neurotrophin receptors, such as TrkA, TrkB, TrkC and/or their signalling and receptor tyrosine kinases, such as FGFR1 and IGF1R and/or their signalling, are beneficial, such as in the treatment and/or prevention of both non-neurological and neurological diseases.

One embodiment relates to the use of a compound of the invention (for example in the manufacture of a pharmaceutical medicament) for use in a method of treating, preventing or reducing the risk of, one or more disease mentioned hereinbefore, which comprises administering to a mammal, such as a human, in need thereof, a therapeutically effective amount of a compound of the invention, or a pharmaceutically acceptable salt thereof.

Another embodiment relates to such a use of a compound of the invention in a method of treating, preventing or reducing the risk of Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and/or other parkinsonian disorders.

A further embodiment relates to such a use of a compound of the invention in a method of treating, preventing or reducing the risk of Alzheimer's disease, Parkinson's disease, Cognitive dysfunction in Schizophrenia, Rett's Syndrome and/or Depression.

A further embodiment relates to such a use of a compound of the invention in a method of treating, preventing or reducing the risk of a disease where enhancement of nerve regeneration is beneficial such as demyelinating diseases, such as multiple sclerosis.

A further embodiment relates to such a use of a compound of the invention in a method of treating, preventing or reducing the risk of spinal cord injury, stroke, hypoxia, ischemia and/or brain injury including traumatic brain injury.

A further embodiment relates to such a use of a compound of the invention in a method of treating or preventing constipation, particularly constipation in Parkinson's disease, slow-transit constipation and opioid-induced constipation.

Another embodiment relates to such a use of a compound of the invention in a method of treating, preventing or reducing the risk of mild cognitive impairment, dementia disorders (including dementia of mixed vascular and degenerative origin, presenile dementia, senile dementia and dementia associated with Parkinson's disease, progressive supranuclear palsy or corticobasal degeneration) and/or cognitive dysfunction in schizophrenia.

A further embodiment relates to such a use of a compound of the invention in a method of treating, obesity, diabetes and metabolic syndrome, diabetic neuropathy including complications thereof such as osteoporosis, painful connective tissue disorders and tendon ruptures, Charcot Marie Tooth disease and its variants, nerve transplantation and its complications, diabetes-induced osteoporosis, motor neuron disease, peripheral nerve injury, genetic or acquired or traumatic hearing loss, blindness and posterior eye diseases, depression, obesity, metabolic syndrome and/or pain.

A further embodiment relates to such a use of a compound of the invention in a method of treating, obesity, diabetes and metabolic syndrome, diabetic neuropathy including complications thereof such as osteoporosis, painful connective tissue disorders and tendon ruptures, Charcot Marie Tooth disease and its variants, nerve transplantation and its complications, motor neuron disease, peripheral nerve injury, genetic or acquired or traumatic hearing loss, blindness and posterior eye diseases, depression, obesity, metabolic syndrome and/or pain.

Yet another embodiment relates to such a use of a compound of the invention in a method of treating, preventing or reducing the risk of depression, schizophrenia and/or anxiety.

As described above, treatment of the disorders described herein with the compounds of the invention may be particularly effective in patients with the Val66Met mutation in the BDNF gene. Accordingly, in particular embodiments, the treatment of the disorders characterised by impaired signalling of neurotrophins and/or other trophic factors as defined herein (including the various embodiments described herein) is in a patient with the Val66Met mutation in the BDNF gene.

Pharmaceutical Compositions

As described herein, compounds of the invention are useful as pharmaceuticals. Such compounds may be administered alone or may be administered by way of known pharmaceutical compositions/formulations.

In a further aspect of the invention, there is provided a pharmaceutical composition comprising a compound of the invention, or a pharmaceutically-acceptable salt thereof, and a pharmaceutically-acceptable excipient, such as a pharmaceutically-acceptable adjuvant, diluent or carrier, for use in the treatment of a disease characterised by impaired signalling of neurotrophins and/or other trophic factors (including the various diseases and disorders listed herein), optionally in a patient with the Val66Met mutation in the BDNF gene.

As used herein, the term pharmaceutically-acceptable excipients includes references to vehicles, adjuvants, carriers, diluents, pH adjusting and buffering agents, tonicity adjusting agents, stabilizers, wetting agents and the like. In particular, such excipients may include adjuvants, diluents or carriers.

The skilled person will understand that compounds of the invention may act systemically and/or locally (i.e. at a particular site), and may therefore be administered accordingly using suitable techniques known to those skilled in the art.

The skilled person will understand that compounds and compositions as described herein will normally be administered orally, intravenously, subcutaneously, buccally, rectally, dermally, nasally, tracheally, bronchially, sublingually, intranasally, topically (including topical administration to the eyes), by any other parenteral route or via inhalation, in a pharmaceutically acceptable dosage form.

Conventional procedures for the selection and preparation of suitable pharmaceutical formulations are described in, for example, “Pharmaceuticals—The Science of Dosage Form Designs”, M. E. Aulton, Churchill Livingstone, 1988. For preparing pharmaceutical compositions from the compounds of the invention, inert, pharmaceutically acceptable carriers can be either solid or liquid. Solid form preparations include powders, tablets, dispersible granules, capsules, cachets, and suppositories.

Pharmaceutical compositions as described herein will include formulations in the form of tablets, capsules or elixirs for oral administration, suppositories for rectal administration, sterile solutions or suspensions for parenteral or intramuscular administration, and the like. Alternatively, particularly where such compounds of the invention act locally, pharmaceutical compositions may be formulated for topical administration. In particular, compounds may be formulated for local delivery to the CNS, for example in the form of artificial cerebrospinal fluid (CSF).

Thus, in particular embodiments, the pharmaceutical composition is provided in a pharmaceutically acceptable dosage form, including tablets or capsules, liquid forms to be taken orally or by injection, suppositories, creams, gels, foams, inhalants (e.g. to be applied intranasally), or forms suitable for topical administration. For the avoidance of doubt, in such embodiments, compounds of the invention may be present as a solid (e.g. a solid dispersion), liquid (e.g. in solution) or in other forms, such as in the form of micelles.

Thus, compounds, of the present invention, and compositions comprising the same, may be administered orally, parenterally, buccally, vaginally, rectally, by inhalation, by insufflation, sublingually, intramuscularly, subcutaneously, topically (including topical administration to the eyes), intranasally, intraperitoneally, intrathoracically, intravenously, epidurally, intrathecally, intracerebroventricularly and by injection into the joints.

Depending on the mode of administration, pharmaceutical compositions will preferably comprise from 0.05 to 99%wt (per cent by weight), more preferably from 0.05 to 80%wt, still more preferably from 0.10 to 70%wt, and even more preferably from 0.10 to 50%wt, of a compounds of the invention (calculated as a non-salt form), all percentages by weight being based on total composition.

Depending on e.g. potency and physical characteristics of the compound of the invention (i.e. active ingredient), pharmaceutical formulations that may be mentioned include those in which the active ingredient is present in an amount that is at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The quantity of the compound to be administered will vary for the patient being treated and will vary from about 100 ng/kg of body weight to 100 mg/kg of body weight per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in uses or methods of the invention.

More, particularly, the skilled person will understand that compounds of the invention may be administered (for example, as formulations as described hereinbefore) at varying doses, with suitable doses being readily determined by one of skill in the art. Oral, pulmonary and topical dosages (and subcutaneous dosages, although these dosages may be relatively lower) may range from between about 0.01 μg/kg of body weight per day (pg/kg/day) to about 14 mg/kg/day, preferably about 0.01 μg/kg/day to about 10 mg/kg/day, and more preferably about 0.1 μg/kg/day to about 5.0 mg/kg/day. For example, when administered orally, treatment with such compounds may comprise administration of a formulations typically containing between about 0.01 μg to about 1000 mg, for example between about 0.1 μg to about 500 mg, or between 1 μg to about 100 mg (e.g. about 20 μg to about 80 mg), of the active ingredient(s). When administered intravenously, the most preferred doses will range from about 0.001 to about 10 μg/kg/hour during constant rate infusion. Advantageously, treatment may comprise administration of such compounds and compositions in a single daily dose, or the total daily dosage may be administered in divided doses of two, three or four times daily (e.g. twice daily with reference to the doses described herein, such as a dose of 1 mg, 5 mg, 10 mg, 25 mg, 50 mg, 100 mg or 200 mg twice daily).

For the avoidance of doubt, the skilled person (e.g. the physician) will be able to determine the actual dosage which will be most suitable for an individual patient, which is likely to vary with the route of administration, the type of formulation, the type and severity of the condition that is to be treated, other medication the patient may be taking, as well as the species, age, weight, size, sex, diet, renal function, hepatic function, general physical condition, genetic factors and response of the particular patient to be treated. Although the above-mentioned dosages are exemplary of the average case, there can, of course, be individual instances where higher or lower dosage ranges are merited, and such doses are within the scope of the invention.

Thus, in a further aspect of the invention, there is provided a use of a pharmaceutical composition, as defined above, in therapy, or for the treatment and/or prevention of a disease characterised by impaired signalling of neurotrophins and/or other trophic factors.

Combinations and Kits-of-Parts

The treatment and/or prevention of diseases of the nervous system and related pathologies defined herein may comprise administration of a compound of the invention as a sole therapy or may involve, in addition to the compound of the invention, conjoint treatment with conventional therapy of value in treating one or more disease conditions referred to herein. Such conventional therapy may include one or more agents such as acetyl cholinesterase inhibitors, anti-inflammatory agents, cognitive and/or memory enhancing agents, atypical antipsychotic agents, dopamine agonists and/or L-DOPA.

Such conjoint treatment and/or prevention may be achieved by way of the simultaneous, sequential or separate dosing of the individual compounds of the invention or additional agents of the treatment and/or prevention. Such combination products employ the compounds, or pharmaceutically-acceptable salts thereof, of the invention.

Accordingly, the skilled person will understand that treatment with compounds of the invention may further comprise (i.e. be combined with) further treatment(s) or preventative methods for the same condition. In particular, treatment with compounds of the invention may be combined with means for the treatment of diseases characterised by impaired signalling of neurotrophins and/or other trophic factors (such as Alzheimer's disease, Parkinson's disease, cognitive dysfunction and depression as described herein, e.g. Alzheimer's disease) such as treatment with one or more other therapeutic agent that is useful in the in the treatment the various diseases characterised by impaired signalling of neurotrophins and/or other trophic factors described herein, and/or one or more physical method used in the treatment (such as treatment through surgery), as known to those skilled in the art.

As described herein, compounds of the invention may also be combined with one or more other (i.e. different) therapeutic agents (i.e. agents that are not compounds of the invention) that are useful in the treatment and/or prevention of diseases characterised by impaired signalling of neurotrophins and/or other trophic factors. Such combination products that provide for the administration of a compound of the invention in conjunction with one or more other therapeutic agent may be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the one or more other therapeutic agent).

Thus, according to a further aspect of the invention, there is provided a combination product comprising:

(I) a compound of the invention as hereinbefore defined, or a pharmaceutically acceptable salt thereof; and

(II) one or more other therapeutic agent that is useful in the treatment or prevention of a disease characterised by impaired signalling of neurotrophins and/or other trophic factors,

wherein each of components (I) and (II) is formulated in admixture, optionally with a pharmaceutically-acceptable excipient, such as a pharmaceutically-acceptable adjuvant diluent or carrier.

According to a further aspect of the invention there is provided a pharmaceutical composition comprising a compound of the invention as hereinbefore defined, or a pharmaceutically acceptable salt thereof and one or more other therapeutic agent that is useful in the treatment or prevention of a disease characterised by impaired signalling of neurotrophins and/or other trophic factors, fomulated together in admixture, optionally with a pharmaceutically-acceptable excipient, such as a pharmaceutically-acceptable adjuvant diluent or carrier.

According to a further aspect of the invention, there is provided a kit-of-parts comprising:

(a) a pharmaceutical composition comprising a compound of the invention as hereinbefore defined, or a pharmaceutically acceptable salt thereof, formulated in admixture, optionally with a pharmaceutically-acceptable excipient, such as a pharmaceutically-acceptable adjuvant diluent or carrier; and

(b) a pharmaceutical composition comprising one or more other therapeutic agent that is useful in the treatment or prevention of a disease characterised by impaired signalling of neurotrophins and/or other trophic factors, formulated in admixture, optionally with a pharmaceutically-acceptable excipient, such as a pharmaceutically-acceptable adjuvant diluent or carrier,

which components (a) and (b) are each provided in a form that is suitable for administration in conjunction with the other.

With respect to the kits-of-parts as described herein, by “administration in conjunction with” (and similarly “administered in conjunction with”) we include that respective formulations are administered, sequentially, separately or simultaneously, as part of a medical intervention directed towards treatment of the relevant condition.

Thus, in relation to the present invention, the term “administration in conjunction with” (and similarly “administered in conjunction with”) includes that the two active ingredients are administered (optionally repeatedly) either together, or sufficiently closely in time, to enable a beneficial effect for the patient, that is greater, over the course of the treatment and/or prevention of the relevant condition, than if either agent is administered (optionally repeatedly) alone, in the absence of the other component, over the same course of treatment and/or prevention. Determination of whether a combination provides a greater beneficial effect in respect of, and over the course of, treatment or prevention of a particular condition will depend upon the condition to be treated or prevented, but may be achieved lo routinely by the skilled person.

Further, in the context of the present invention, the term “in conjunction with” includes that one or other of the two formulations may be administered (optionally repeatedly) prior to, after, and/or at the same time as, administration of the other component. When used in this context, the terms “administered simultaneously” and “administered at the same time as” includes instances where the individual doses of the compound of the invention and the additional compound for the treatment of a disease characterised by impaired signalling of neurotrophins and/or other trophic factors, or pharmaceutically acceptable salts thereof, are administered within 48 hours (e.g. within 24 hours, 12 hours, 6 hours, 3 hours, 2 hours, 1 hour, 45 minutes, 30 minutes, 20 minutes or 10 minutes) of each other.

Other therapeutic agents useful in the treatment or prevention of diseases characterised by impaired signalling of neurotrophins and/or other trophic factors will be well-known to those skilled in the art. For example, such other therapeutic agents may include: acetyl cholinesterase inhibitors, anti-inflammatory agents, cognitive enhancing agents, memory enhancing agents, and atypical antipsychotic agents, anti-depressive agents, anti-Alzheimer's agents, beta-secretase inhibitors, gamma-secretase modulators, agents modifying tau function, amyloid-beta production inhibitors, antibodies directed at amyloid-beta, antibodies directed at tau, antibodies directed at alpha-synuclein, anti-Parkinson agents, anti-diabetic agents, anti-multiple sclerosis agents, anti-obesity agents, agents used for treatment of auditory dysfunction, agents used for treatment of ocular disease, agents used for the treatment of olfactory dysfunction, agents used for the treatment of gustatory dysfunction, anti-Huntington agents, anti-Rett syndrome agents, anti-stroke agents. Particular therapeutic agents that may be mentioned include acetyl cholinesterase inhibitors, anti-Alzheimer's agents, anti-Parkinson agents, cognitive enhancing agents, antibodies directed at amyloid-beta, antibodies directed at tau, antibodies directed at alpha-synuclein, beta-secretase inhibitors and gamma-secretase modulators, anti-constipation agents (such as laxatives, serotonin agonists and chloride channel activators).

Preparation of Compositions

Pharmaceutical compositions/formulations, combination products and kits as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a compound of the invention, as hereinbefore defined, with one or more pharmaceutically-acceptable excipient.

In further aspects of the invention, there is provided a process for the preparation of a combination product or kit-of-parts as hereinbefore defined, which process comprises bringing into association a compound of the invention, as hereinbefore defined, with the other therapeutic agent that is useful in the treatment of the relevant disease or disorder, and at least one pharmaceutically-acceptable excipient.

As used herein, references to bringing into association will mean that the two components are rendered suitable for administration in conjunction with each other.

Thus, in relation to the process for the preparation of a kit-of-parts as hereinbefore defined, by bringing the two components “into association with” each other, we include that the two components of the kit-of-parts may be:

(i) provided as separate formulations (i.e. independently of one another), which are subsequently brought together for use in conjunction with each other in combination therapy; or

(ii) packaged and presented together as separate components of a “combination pack” for use in conjunction with each other in combination therapy.

Preparation of Compounds of the Invention

Compounds of the invention as described herein may be prepared either as a free base or as a pharmaceutically acceptable salt in accordance with techniques that are well known to those skilled in the art, such as those described in the examples provided hereinafter.

According to a further aspect of the invention there is provided a process for the preparation of a compound of the invention, which comprises the step of reaction of a compound of formula II,

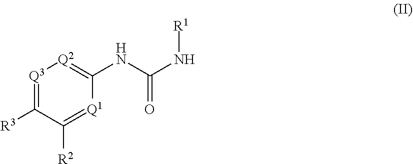

wherein R¹, R², R³, Q¹, Q² and Q³ are as hereinbefore defined, with ethoxycarbonyl isocyanate.

This reaction may be performed for example:

a. in a sealed microwave vial in a suitable solvent (such as toluene or bromobenzene), at a suitable reaction temperature (e.g. between room temperature and reflux temperature); or

b. by first treating the compound of formula II with a suitable base, such as sodium hydride, at a suitable reaction temperature (e.g. between 0° C. and room temperature) for between about 1 and about 60 minutes in a suitable solvent, such as DMF, followed by the addition of the ethoxycarbonyl isocyanate at approximately the same, with stirring, for a suitable time, such as between about 1 and about 60 minutes.

Compounds of formula II may be obtained by reacting a compound of formula III,

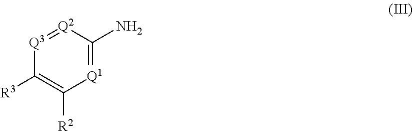

wherein R², R³, Q¹, Q² and Q³ are as hereinbefore defined, with either:

a. a compound of formula IV,

R¹—N═C═O (IV)

wherein R¹ is as hereinbefore defined; or

(b) a compound of formula V,

R¹—N(H)C(O)Cl (V)

wherein R¹ is as hereinbefore defined, for example (in both cases) in the presence of a suitable base, such as TEA, in a suitable solvent, such as DCM, THF or, pyridine, at a suitable reaction temperature (for example between room temperature and reflux temperature).

Compounds of formula II may alternatively be obtained by reacting a compound of formula VI,

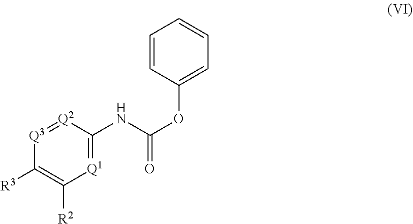

wherein R², R³, Q¹, Q² and Cr are as hereinbefore defined, with an amine of formula VII,

R¹—NH₂ (VII)

wherein R¹ is as hereinbefore defined, for example in the presence of a suitable base, such as TEA, in a suitable solvent, such as THF, and at a suitable reaction temperature (e.g. between room temperature and reflux temperature).

Compounds of formula II may alternatively be prepared by reacting a compound of formula III as hereinbefore defined with triphosgene or phosgene in the presence of a suitable base, such as NaHCO₃ or TEA, in a suitable solvent, such as DCM, and at a suitable reaction temperature (e.g. between 0° C. and room temperature). After a suitable period of time, such as between about 1 and about 6 hours, a compound of formula VII may be added, together with an additional amount of a suitable (e.g. the above-mentioned) base, which reaction mixture is then allowed to react at a suitable temperature, such as room temperature, for a suitable period of time, such as between about 1 and about 24 hours. Alternatively, the sequence of this reaction may be altered by first reacting a compound of formula IV with triphosgene or phosgene, followed by the addition of the compound of formula VII, under substantially the same reaction conditions as described above.

Compounds of formula III may be obtained by reducing a compound of formula VIII,

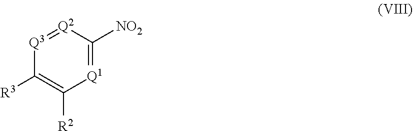

wherein R², R³, Q¹, Q² and Cr are as hereinbefore defined, in the presence of a suitable reducing agent such as SnCl₂.2H₂O, for example in the presence of HCl, or using Pd/C in the presence of H₂(g). This reaction may be performed in a suitable solvent, such as ethanol, and at a suitable temperature (for example between room temperature and reflux temperature).

In a further embodiment of the invention, there is provided a process for the preparation of a compound of the invention from a compound of formula IX,

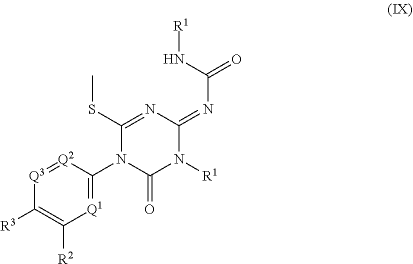

wherein R¹, R², R³, Q¹, Q² and Cr are as hereinbefore defined, particularly wherein R¹ represents a phenyl or heteroaryl group as defined herein (more particularly wherein R¹ represents phenyl or tolyl), in the presence of a suitable acid (such as HCl (e.g. 2M HCl)) and optionally an organic co-solvent (e.g. 1,4-dioxane), and at a suitable temperature (for example at between room temperature and reflux temperature).

Compounds of formula IX may be obtained by reacting a compound of formula X

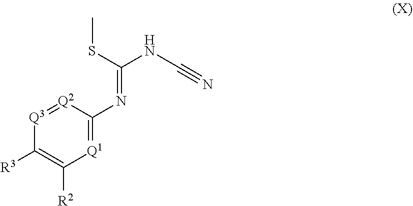

wherein wherein R², R³, Q¹, Q² and Cr are as hereinbefore defined, particularly wherein R¹ represents an phenyl or heteroaryl group as defined herein (more particularly wherein

R¹ represents phenyl or tolyl), with an excess (e.g. 2 equivalents) of a compound of formula XI,

R¹—N═C═O (XI)

wherein R¹ is as hereinbefore defined, particularly wherein R¹ represents an aryl or heteroaryl group as defined herein (more particularly wherein R¹ represents phenyl), in the presence of a suitable base (e.g triethylamine) a suitable solvent (e.g. acetonitrile) and a suitable reagent (e.g. 1,1′-Carbonyldiimidazole (ODD), at a suitable temperature (e.g, room temperature).

Compounds of formula X may be obtained by reacting a compound of formula III as defined herein with dimethyl N-cyanodithioiminocarbonate in the presence of a suitable solvent (e.g. ethanol) at a suitable temperature (e.g. reflux temperature) for a suitable period of time (e.g. an extended period of time, such as more than 24 hours (e.g. more than 48 hours).

Compounds of formulae IV, V, VI, VII,VIII and XI are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

The skilled person will understand that the substituents as defined herein, and substituents thereon, may be modified one or more times, after or during the processes described above for the preparation of compounds of the invention by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention.

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well-known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999), the contents of which are incorporated herein by reference.

When used herein in relation to a specific value (such as an amount), the term “about” (or similar terms, such as “approximately”) will be understood as indicating that such values may vary by up to 10% (particularly, up to 5%, such as up to 1%) of the value defined. It is contemplated that, at each instance, such terms may be replaced with the notation “±10%”, or the like (or by indicating a variance of a specific amount calculated based on the relevant value). It is also contemplated that, at each instance, such terms may be deleted.

Compounds of the invention may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, compounds of the invention may have the advantage that they are more efficacious and/or exhibit advantageous properties in vivo.

### EXAMPLES

The present invention will be further described by reference to the following examples, which are not intended to limit the scope of the invention.

Experimental Procedures

Starting materials and intermediates used in the synthesis of compounds described herein are commercially available or can be prepared by the methods described herein or by methods known in the art.

Experiments were generally carried out under inert atmosphere (nitrogen or argon), particularly in cases where oxygen- or moisture-sensitive reagents or intermediates were used.

Mass spectrometry data are reported from liquid chromatography-mass spectrometry (LC-MS) using electrospray ionization. Chemical shifts for NMR data are expressed in parts per million (ppm, 6) referenced to residual peaks from the deuterated solvent used.

For syntheses referencing general procedures, reaction conditions (such as length of reaction or temperature) may vary. In general, reactions were followed by thin layer chromatography or LC-MS, and subjected to work-up when appropriate. Purifications may vary between experiments: in general, solvents and the solvent ratios used for eluents/gradients were chosen to provide an appropriate R_{f} and/or retention time.

General Methods

All solvents were of analytical grade and commercially available anhydrous solvents were routinely used for reactions. Starting materials used were available from commercial sources or prepared according to literature procedures, Room temperature refers to 20-25° C. Solvent mixture compositions are given as volume percentages or volume ratios.

MW heating was performed in a standard MW reactor producing continuous irradiation at 2450 MHz. It is understood that MWs can be used for the heating of reaction mixtures.

Typically, an Anton paar microwave synthesizer 300 was used as a microwave synthesizer.

Thin layer chromatography (TLC) was performed on Merck TLC-plates (Silica gel 60 F₂₅₄) and spots were UV visualized. TLC was generally used to monitor reaction progression and solvents used were for example: ethyl acetate or acetonitrile or DCM with 1-10% of MeOH, ethyl acetate with 0-95% hexane. Straight phase flash column chromatography (“flash chromatography”/“column chromatography”) was manually performed on Merck Silica gel 60 (0.040-0.063 mm) or basic aluminum oxide or neutral aluminum oxide, or automatically using ISCO Combiflash® Companion™ system using RediSep™ normal-phase flash columns (“Combiflash”) using the solvent system indicated.

NMR spectra was recorded on a 400 MHz NMR spectrometer (Bruker 400 MHz Avance-III) fitted with a probe of suitable configuration. Spectra were recorded at ambient temperature unless otherwise stated. Chemical fields are given in ppm down- and upfield from TMS (0.00 ppm). The following reference signals were used in ¹H-NMR: TMS a 0.00, or residual solvent signal of DMSO-d6 δ 2.49, CDCl₃ δ7.25 (unless otherwise indicated). Resonance multiplicities are denoted s, d, t, q, m, dd, tt, dt br and app for singlet, doublet, triplet, quartet, doublet of doublet, triplet of triplet, doublet of triplet, multiplet, broad and apparent, respectively. In some cases only diagnostic signals are reported.

High pressure liquid chromatography (HPLC) was performed on a reversed phase (RP) column. A gradient was applied using for example mobile phase A (5 mM Ammonium acetate +0.1% Formic acid in water) and B (0.1% Formic acid in Acetonitrile) or A (0.1% NH3 in water) and B (0.1% NH3 in acetonitrile) or A (10 mM Ammonium actetate in water) and B (Acetonitrile).

Reversed phase columns used were for example: BEH C18 (50*2.1 mm), 1.7 μm; X-Bridge C18 (50*4.6 mm), 3.5 μm; X-Bridge/YMCC18 (150*4.6 mm), 5 μm; BEH C18 (50*2.1 mm), 1.7 μm; X-Bridge C8 (250*19) mm, 5 μm. The flowrate used was for example 0.55 ml/min or 1.00 ml/min

Mass spectrometry (MS) analysis were performed in positive and/or negative ion mode using electrospray ionization (ESI+/−).

Preparative HPLC chromatography was run on a Waters e2695 Separation Module with a PDA Detector or on a Shimadzu LC-20AP with an UV detector. Column; X-BRIDGE C18, 150*4.6 mm, 5 μm or X-Bridge C18 (250*19 mm) 5 μm or GEMINI C18 (250*21.2 mm) 5 μm or sunfire c18(150*19)mm, 5 micron or x-bridge c18(150*19)mm, 5 micron or ymc actus triart c18(150*20)mm, 5 micron or kromasil eternity c18 (250*21.2)mm, 5 micron. The flowrate used was for example 10-15 ml/min. The UV spectra were typically recorded at 202 nm & between 214 and 260 nm Lambda max.

A gradient was applied using for example mobile phase A (0.1% NH₃ in water) and B (0.1% NH3 in acetonitrile); A (0.1% TFA in water) and B (Acetonitrile); A (5 mM ammonium bicarbonate +0.05% ammonia in water) and B (Acetonitrile); A (5 mM ammonium bicarbonate) and B (acetonitrile) for LC-separation at a flow rate 1 ml/min.

High pressure liquid chromatography (HPLC) was performed on a straight phase column. A linear gradient or isocratic flow was applied using for example phase A (Hexane) and B (XX)

Compounds have been named using CDD vault from Collaborative Drug Discovery Inc. Burlingame Calif., USA or ChemDoodle 8.1.0/9.02 from iChemLabs LLC, USA or ACD/ChemSketch 2012 (14.01) from Advanced Chemistry Development (ACD/labs) Ontario, Canada. In case of inconsistency between a name of a compound and the structural formula of the same compound, it is the structural formula that is decisive for the molecular structure of the compound.

In the event that there is a discrepancy between nomenclature and any compounds depicted graphically, then it is the latter that presides (unless contradicted by any experimental details that may be given or unless it is clear from the context).

### Intermediate 1

### 1-(2-methylbiphenyl-4-yl)-3-phenylurea

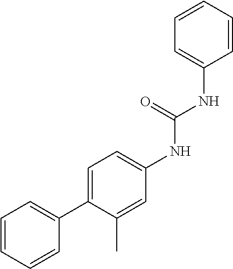 [SMILES: Cc1cc(NC(=O)Nc2ccccc2)ccc1-c1ccccc1]

2-methylbiphenyl-4-amine (commercially available, 0.60 g, 3.2 mmol) was added to a RBF previously equipped with a magnetic stirrer. DCM (12 ml) and TEA (1.38 ml, 9.8 mmol) were added and the mixture was cooled to 0° C. Phenyl Isocyanate (0.46 g, 3.9 mmol) was added and the resulting reaction mixture was allowed to reach 25° C. and stirred for 16 h. The obtained solid precipitate was filtered out and washed with n-pentane (20 ml) to obtain 0.55 g (55% yield) of the title compound. ¹H NMR (400 MHz, DMSO-d₆): δ8.69 (s, 1H), 8.68 (s, 1H), 7.48-7.37 (m, 6H), 7.35-7.27 (m, 5H), 7.11 (d, J=8.4 Hz, 1H), 6.98 (t, J=7.2 Hz, 1H), 2.22 (s, 3H); MS (ES+) m/z 303 [M+H]⁺

### Intermediate 2

### 1-[3-methyl-4-(piperidin-1-yl)phenyl]-3-phenylurea

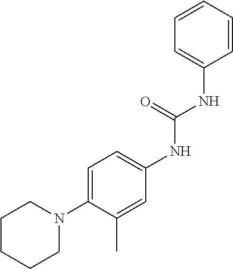 [SMILES: Cc1cc(NC(=O)Nc2ccccc2)ccc1N1CCCCC1]

To a RBF, previously equipped with a magnetic stirrer and nitrogen balloon, 3-methyl-4-(piperidin-1-yl)aniline (commercially available, 0.33 g, 1.7 mmol) and TEA (0.36 ml, 2.6 mmol) in DCM (3.3 ml) were added and the mixture was cooled to 0° C. Phenyl isocyanate (0.20 g, 1.7 mmol) was added and the resulting reaction mixture was allowed to slowly reach 25° C. and stirred for 2 h. The obtained solid precipitate were filtered out and washed with n-pentane (30 ml) to yield 0.390 g (72% yield) of the title compound. ¹H NMR (400 MHz, DMSO-d₆): δ8.58 (s, 1H), 8.43 (s, 1H), 7.44 (d, J=7.6 Hz, 2H), 7.28-7.18 (m, 4H), 6.96-6.92 (m, 2H), 2.73 (t, J=5.2 Hz, 4H), 2.22 (s, 3H), 1.63-1.51 (m, 6H). MS (ES+) m/z 310 [M+H]⁺

### Intermediate 3

### 1[3-methyl-4-(prop-1-en-2-yl)phenyl]-3-phenylurea

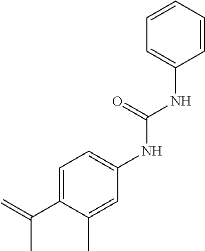 [SMILES: C=C(C)c1ccc(NC(=O)Nc2ccccc2)cc1C]

In a microwave vial previously equipped with a magnetic stirrer and argon balloon was added 1-(4-bromo-3-methylphenyl)-3-phenylurea (commercially available, 1.00 g, 3.2 mmol), potassium isopropenyltrifluoroborate (0.58 g, 3.9 mmol) and TEA (1.38 ml, 9.8 mmol) in isopropyl alcohol (10 ml) and H₂O (10 ml) at RT. The reaction mixture was purged with argon gas for 15 min and Pd(dppf)C1₂.DCM (0.13 g, 0.1 mmol) was added. The reaction mixture was stirred at 85° C. for 3 h. The reaction mixture was filtered through celite bed and washed with ethyl acetate (50 ml). Water (10 ml) was added to the filtrate and the product was extracted with ethyl acetate (2×25 ml). The combined organic layers were washed with brine (25 ml), dried over sodium sulphate and evaporated under reduced pressure. The crude product was purified by combi flash chromatography using 10% ethyl acetate in hexanes as an eluent to obtain 0.700 g (80% yield) of the title compound. ¹H NMR (400 MHz, DMSO-d₆): δ8.64 (s, 1H), 8.58 (s, 1H), 7.47 (d, J=7.6 Hz, 2H), 7.30-7.23 (m, 4H), 7.04 (d, J=8.0 Hz, 1H), 6.97 (t, J=7.6 Hz, 1H), 5.20 (s, 1H), 4.81 (s, 1H), 2.26 (s, 3H), 2.00 (s, 3H); MS (ES+) m/z 267 [M+H]⁺

### Intermediate 4

### 1-[3-methyl-4-(propan-2-yl)phenyl]-3-phenylurea

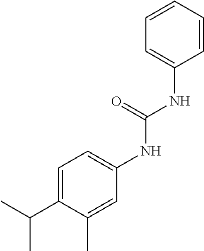 [SMILES: Cc1cc(NC(=O)Nc2ccccc2)ccc1C(C)C]

In a RBF previously equipped with a magnetic stirrer and nitrogen balloon was added 1-[3-methyl-4-(prop-1-en-2-yl)phenyl]-3-phenylurea (Intermediate 3, 0.66 g, 8 mmol) and Ethanol (13.2 ml). Pd/C (10%, 50% wet, 0.06 g) was added to the stirred solution under N2 atmosphere. The reaction mixture was stirred for 5 h under H2 (gas) atmosphere.The reaction mixture was filtered through celite bed and washed with ethyl acetate. The solvent was evaporated under reduced pressure to obtain 0.620 g (93% yield) of the title compound. ¹ H NMR (400 MHz, DMSO-d₆): δ8.61 (s, 1H), 8.48 (s, 1H), 7.45 (d, J=7.6 Hz, 2H), 7.29-7.20 (m, 4H), 7.13 (d, J=8.0 Hz, 1H), 6.96 (t, J=7.2 Hz, 1H), 3.07-3.03 (m, 1H), 2.27 (s, 3H), 1.16 (d, J=6.8 Hz, 6H); MS (ES+) m/z 269 [M+H]⁺

### Intermediate 5

### 1-[3-methyl-4-(5-methylfuran-2-yl) phenyl]-3-phenyl urea

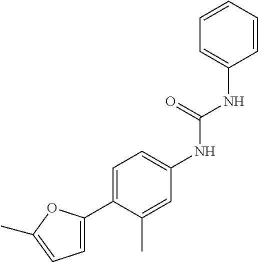 [SMILES: Cc1ccc(-c2ccc(NC(=O)Nc3ccccc3)cc2C)o1]

Into a microwave vial previously equipped with a magnetic stirrer and argon balloon was added 1-(4-bromo-3-methylphenyl)-3-phenylurea (commercially available, 0.50 g, 1.6 mmol) and 5-Methyl-2-furanboronic acid pinacol ester (1.36 g, 6.5 mmol) in ethyl acetate (20 ml) at room temperature. Na₂CO₃ (aq., 2 M, 12.5 ml) was added drop wise and the reaction mixture was purged with argon gas for 15 min. Pd(dppf)Cl₂.DCM (0.13 g, 0.1 mmol) was added and the reaction mixture was stirred at 70° C. for 16 h. The reaction mixture was filtered through a celite bed and washed with ethyl acetate (50 ml) and to the filtrate water (25 ml) was added. The organic layer was separated and the aqueous layer was extracted with additional amount of ethyl acetate (2×25 ml). The combined organic layer was washed with brine (25 ml), dried over sodium sulphate and concentrated under reduced pressure. The crude product was purified by combi flash chromatography using 20% ethyl acetate in hexanes as an eluent to obtain 0.450 g (89% yield) of the title compound. ¹H NMR (400 MHz, DMSO-d₆): δ8.71 (s, 1H), 8.69 (s, 1H), 7.54 (d, J=8.4 Hz, 1H), 7.47 (d, J=8.0 Hz, 2H), 7.39-7.35 (m, 2H), 7.29 (t, J=7.8 Hz, 2H), 6.98 (t, J=7.3 Hz, 1H), 6.48 (d, J=3.2 Hz, 1H), 6.20 (d, J=3.1 Hz, 1H), 2.41 (s, 3H), 2.35 (s, 3H); MS (ES+) m/z 307 [M+H]⁺

### Intermediate 6

### 1-(4′-ethyl-2-methylbiphenyl-4-yl)-3-methylurea

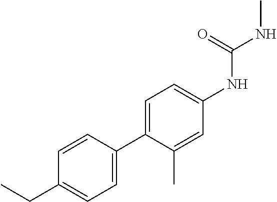 [SMILES: CCc1ccc(-c2ccc(NC(=O)NC)cc2C)cc1]

4′-ethyl-2-methylbiphenyl-4-amine (commercially available, 0.54 g, 2.5 mmol) and TEA (0.71 ml, 5.1 mmol) in DCM (10.8 ml) was added to a RBF previously equipped with a magnetic stirrer and nitrogen balloon and cooled to 0° C. Methylaminoformyl chloride (0.28 g, 3 mmol) was added and the reaction mixture was allowed to reach 25° C. and stirred for 16 h. The obtained solid precipitates were filtered out and washed with n-pentane (20 ml) to yield 0.8 g (quantitative yield) of the title compound. MS (ES+) m/z 269 [M+H]⁺

### Intermediate 7

### 1-[3-methyl-4-(1H-pyrrol-1-yl)phenyl]-3-phenylurea

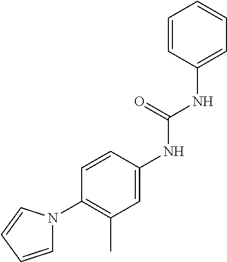 [SMILES: Cc1cc(NC(=O)Nc2ccccc2)ccc1-n1cccc1]

3-methyl-4-(1H-pyrrol-1-yl)-aniline (commercially available, 1.00 g, 5.8 mmol) and TEA (1.63 ml, 11.6 mmol) in DCM (10 ml) was added to a RBF previously equipped with a magnetic stirrer and nitrogen balloon and the mixture was cooled to 0° C. Phenyl isocyanate (0.69 g, 5.8 mmol) was added and the reaction mixture was allowed to reach 25° C. gradually and was stirred for 2 h. The obtained solid precipitates were filtered out and washed with n-pentane (30 ml) to yield 1.0 g (59% yield) of the title compound. MS (ES+) m/z 292 [M+H]⁺

### Intermediate 8

### 1-methyl-3-(2-methylbiphenyl-4-yl)urea

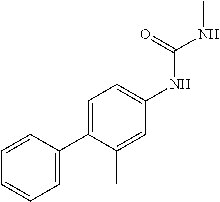 [SMILES: CNC(=O)Nc1ccc(-c2ccccc2)c(C)c1]

Into a RBF, previously equipped with a magnetic stirrer and nitrogen balloon, was added 2-methylbiphenyl-4-amine (commercially available, 0.50 g, 2.7 mmol) and TEA (0.76 ml, 5.4 mmol) in DCM (10.0 ml) and the mixture was cooled to 0° C. Methylaminoformyl chloride (0.30 g, 3.2 mmol) was added and the resulting reaction mixture was allowed to reach 25° C. and was stirred for 16 h. The obtained solid precipitates were filtered out and washed with n-pentane (20 ml) to yield 0.75 g of the title compound that was directly used in the next step without further purification.

### Intermediate 9

### 3-[3-methyl-4-(1H-pyrazol-1-yl)phenyl]-1-phenylurea

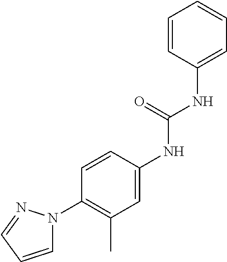 [SMILES: Cc1cc(NC(=O)Nc2ccccc2)ccc1-n1cccn1]

To a RBF, previously equipped with a magnetic stirrer and nitrogen bubbler, 3-methyl-4-(1H-pyrazol-1-yl)aniline (0.8 g, 4.61 mmol, commercially available) in DCM (8.0 ml) was added. The mixture was cooled to 0° C. and triethylamine (0.64 ml, 4.61 mmol) was added followed by the addition of Phenyl Isocyanate (0.55 g, 4.61 mmol) and the reaction mixture was stirred for 45 min. The reaction mixture was quenched with ice-water (30 ml) and product was extracted with DCM (3×40 ml). The combined organic layer was dried over sodium sulphate and the solvent removed under reduce pressure. The crude product was purified by column chromatography using Hexanes: ethyl acetate as an eluent using 60-120 mesh silica gel to yield 0.6 g (44%) of the title compound. ¹H NMR (400 MHz, DMSO-d₆): δ8.84 (s, 1H), 8.75 (s, 1H), 7.97 (d, J=2.00 Hz, 1H), 7.70 (s, 1H), 7.43-7.41 (m, 4H), 7.27-7.25 (m, 3H), 7.00 (t, J=7.20 Hz, 1H), 6.48 (s, 1H), 2.15 (s, 1H). MS (ES+) m/z 293 [M+H]⁺

### Intermediate 10

### 3-methyl-1-(2-methyl-4-nitrophenyl)-1H-pyrrole

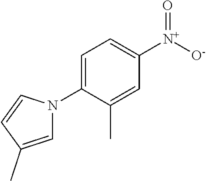 [SMILES: Cc1ccn(-c2ccc([N+](=O)[O-])cc2C)c1]

In a RBF, previously equipped with a magnetic stirrer and nitrogen balloon, was taken 3-Methyl-1H-pyrrole (1.17 g, 14.5 mmol) in DMSO (15 ml) and it was cooled to 0° C. To it, Potassium tert-butoxide (1.62 g, 14.5 mmol) was added portion-wise to the mixture and it was stirred for 1 h. 1-Fluoro-2-methyl-4-nitrobenzene (1.50 g, 9.7 mmol) was added drop-wise to the mixture and the reaction mixture was stirred and allowed to come to room temperature and stirred at room temperature for 1 h. The reaction mixture was quenched with ice-water (50 ml) and product was extracted with Ethyl Acetate (3×30 ml). The combined organic layer was washed with brine (30 ml), dried over sodium sulphate and solvent removed under reduced pressure to obtain 1.4 g (66%) of the title compound. ¹H NMR (400 MHz, DMSO-d₆): δ8.250 (s, 1H), δ8.099 (d, J=8.4, 1H), δ7.459 (d,J=8.8,1H), δ6.971 (s, 1H), δ6.843 (s, 1H), δ6.135(s, 1H), δ2.378 (s, 3H), δ2.081 (s, 3H).

### Intermediate 11

### 3-methyl-4-(3-methyl-1H-pyrrol-1-yl)aniline

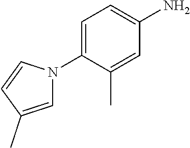

In a RBF previously equipped with a magnetic stirrer and nitrogen balloon were taken_3-methyl-1-(2-methyl-4-nitrophenyl)-1H-pyrrole (Intermediate 10 (1.4 g, 6.47 mmol) and Methanol (21 ml). To the solution 10% Pd/C (50% wet, 0.34 g) was added under nitrogen and reaction mixture was stirred for 6 h under H₂ (gas) bubbling. The reaction mixture was filtered through a celite bed and the bed was washed with Methanol and combined filtrate was concentrated under reduced pressure to obtain 1.0 g (82%) of the title compound. MS (ES+) m/z 187 [M+H]⁺

### Intermediate 12

### N-cyano-N′-[3-methyl-4-(3-methyl-1H-pyrrol-1-yl)phenyl](methylsulfanyl)methanimidamide

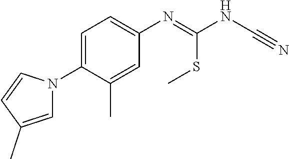 [SMILES: CS/C(=N\c1ccc(-n2ccc(C)c2)c(C)c1)NC#N]

In a sealed tube previously equipped with a magnetic stirrer and nitrogen balloon was taken 3-methyl-4-(3-methyl-1H-pyrrol-1-yl)aniline (Intermediate 11, 0.50 g, 2.68 mmol) in Ethanol (7.5 ml). Dimethyl N-cyanodithioiminocarbonate (0.39 g, 2.68 mmol) was added at room temperature. The reaction mixture was heated to 80° C. for 2 days. The reaction mixture was cooled to room temperature and the solvent was removed under reduced pressure to obtain the crude product that was purified by flash column chromatography using 30% EtOAc in Hexanes as an eluent to obtain 0.35 g (45%) of the title compound. ¹H NMR (400 MHz, DMSO-d₆): δ10.181 (S, 1H), 7.401 (S, 1H), 7.35 (d, J=8.0, 1H), 7.228 (d, J=8.4, 1H), 6.817 (s, 1H), 6.699 (s, 1H), 6.047 (s, 1H), 2.714 (s, 3H), 2.188 (s, 3H), 2.072 (s, 3H). MS (ES+) m/z 285 [M+H]⁺

### Intermediate 13

### 3-[5-[3-methyl-4-(3-methyl-1H-pyrrol-1-yl)phenyl]-1-(3-methylphenyl)-4-(methylsulfanyl)-6-oxo-1,2,5,6-tetrahydro-1,3,5-triazin-2-ylidene]-1-(3-methylphenyl)urea

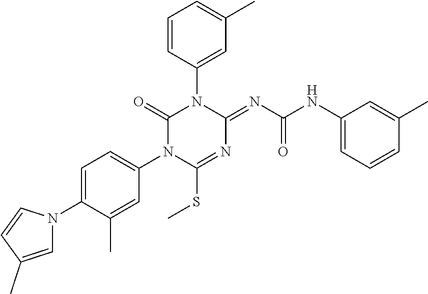 [SMILES: CSc1n/c(=N\C(=O)Nc2cccc(C)c2)n(-c2cccc(C)c2)c(=O)n1-c1ccc(-n2ccc(C)c2)c(C)c1]

In a 25 ml RBF previously equipped with a magnetic stirrer and nitrogen balloon was taken 3-methyl-phenylisocyanate (0.327 g, 2.46 mmol) and N-cyano-N′-[3-methyl-4-(3-methyl-1H-pyrrol-1-yl)phenyl]methylsulfanyl)methanimidamide (Intermediate 12, 0.350 g, 1.23 mmol) in DCM (3.5 ml). To the reaction mixture, triethylamine (1.75 ml) was added followed by addition of CDI (1,1′-Carbonyldiimidazole, 0.598, 3.69 mmol). The reaction mixture was stirred at room temperature for 6 h. The reaction mixture was quenched with water (30 ml) and extracted with DCM (2×50 ml). The combined organic layer was washed with brine (30 ml), dried over sodium sulphate and solvent removed under reduce pressure to obtain 0.450 g crude title compound which was used in the next step without further purification. MS (ES+) m/z 551 [M+H]⁺

### Intermediate 14

### 1-(2-methyl-4-nitrophenyl)-1H-indole

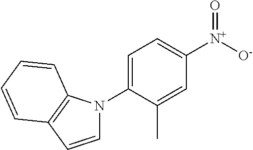 [SMILES: Cc1cc([N+](=O)[O-])ccc1-n1ccc2ccccc21]

In a sealed tube, previously equipped with a magnetic stirrer and nitrogen balloon was taken 1H-Indole (3.7 g, 32.2 mmol) in DMF (30 ml) at room temperature and to it K₂CO₃ (13.3 g, 96.6 mmol) was added. The mixture was stirred for 30 min at room temperature. 1-Fluoro-2-methyl-4-nitrobenzene (5.0 g, 32.2 mmol) in DMF (20 ml) was added and the reaction mixture was stirred at 100° C. for 16 h. The reaction mixture was quenched with ice-water (50 ml) and product was extracted with EtOAc (3×20 ml). The combined organic layer was washed with brine (50 ml), dried over sodium sulphate and the solvent removed under reduced pressure to 5.2 g (63%) of the title compound. ¹H NMR (400 MHz, DMSO-d₆): δ8.40 (s, 1H), 8.23 (d, J=8.8, 1H), 7.69-7.58 (m, 3H), 7.19 -7.08 (m, 3H), 6.75 (d, J=2.8 Hz, 1H), 2.2 (s, 3H).

### Intermediate 15

### 4-(1H-indol-1-yl)-3-methylaniline

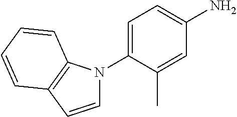 [SMILES: Cc1cc(N)ccc1-n1ccc2ccccc21]

In a RBF previously equipped with a magnetic stirrer and nitrogen balloon was taken 1-(2-methyl-4-nitrophenyl)-1H-indole (Intermediate 14, 5.2 g, 20.6 mmol) in Methanol (50 ml).

To the mixture 10% Pd/C (50% wet, 0.52 g) was added under N₂ atmosphere. The suspension was stirred for 3 h under H₂ (gas) bubbling. The reaction mixture was filtered through celite bed washed with methanol and combined filtrate was concentrated under reduced pressure to obtain 3.5 g (76%) of the title compound which was used without further purification the next step. ¹H NMR (400 MHz, DMSO-d6): δ7.60 (d, J=7.6 Hz, 1H), 7.30 (d, J=2.80 Hz, 1H), 7.10-7.02 (m, 2H), 6.92 (t, J=8.4, 2H), 6.57-6.51 (m, 3H), 5.30 (s,2H), 1.78 (s, 3H); MS (ES+) m/z 223 [M+H]⁺

### Intermediate 16

### 3-[4-(1H-indo1-1-yl)-3-methylphenyl]-1-(3-methylphenyl)urea

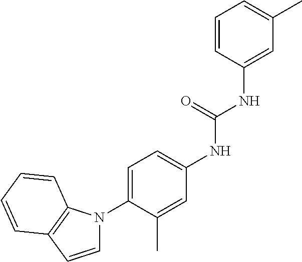 [SMILES: Cc1cccc(NC(=O)Nc2ccc(-n3ccc4ccccc43)c(C)c2)c1]

In a RBF previously equipped with a magnetic stirrer and nitrogen balloon was taken 4-(1H-indol-1-yl)-3-methylaniline (Intermediate 15, 0.50 g, 2.24 mmol), NaHCO₃ (0.567, 6.75 mmol) and DMF (5.0 ml). The mixture was cooled to 0° C., and 3-methyl-phenylisocyanate was added and the reaction mixture was stirred for 2 h at 0° C. The reaction mixture was allowed to reach room temperature and stirred at room temperature for 1 h. The reaction mixture was quenched with water (20 ml) and product was extracted with ethyl acetate (3×20 ml). The combined organic layer was washed with brine (20 ml), dried over sodium sulphate and solvent removed under reduced pressure. The crude product was purified by column chromatography using silica gel (60-120 mesh) and 30% ethyl acetate in hexanes as an eluent to obtain 0.53 g (66%) of the title compound. MS (ES+) m/z 356 [M+H]⁺

### Intermediate 17

### N-cyano-N′-[3-methyl-4-(1H-pyrrol-1-yl)phenyl](methylsulfanyl)methanimidamide

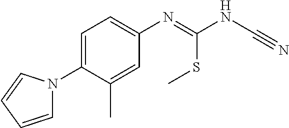 [SMILES: CS/C(=N\c1ccc(-n2cccc2)c(C)c1)NC#N]

In a seal tube previously equipped with a magnetic stirrer and nitrogen balloon was taken 3-methyl-4-(1H-pyrrol-1-yl)-aniline (commercially available, 1.5 g, 8.70 mmol) in Ethanol (22.5 ml). Dimethyl N-cyanodithioiminocarbonate (1.27, 8.70 mmol) was added at room temperature. The reaction mixture was heated to 78° C. for 2 days. The reaction mixture was cooled to room temperature and the solvent was removed under reduced pressure. The crude product was purified by column chromatography using 20% EtOAc in Hexane as an eluent to obtain 1.0 g (42%) of the title compound. MS (ES+) m/z 271 [M+H]⁺

### Intermediate 18

### 3-[5-[3-methyl-4-(1H-pyrrol-1-yl)phenyl]-1-(3-methylphenyl)-4-(methylsulfanyl)-6-oxo-1,2,5,6-tetrahydro-1,3,5-triazin-2-ylidene]-1-(3-methylphenyl)urea

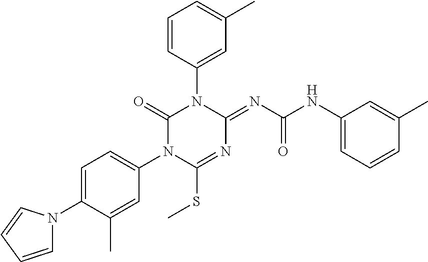 [SMILES: CSc1n/c(=N\C(=O)Nc2cccc(C)c2)n(-c2cccc(C)c2)c(=O)n1-c1ccc(-n2cccc2)c(C)c1]

In a RBF previously equipped with a magnetic stirrer and nitrogen balloon was taken N-cyano-N′-[3-methyl-4-(1H-pyrrol-1-yl)phenyl](methylsulfanyl)methanimidamide (Intermediate 17, 0.90 g, 3.33 mmol) and 3-methyl-phenyl isocyanate (0.88 g, 6.66 mmol) in DCM (9.0 ml). To the reaction mixture, tryethylamine (4.5 ml) was added followed by addition of CDI(1,1′-Carbonyldiimidazole, 1.62 g, 9.99 mmol). The reaction mixture was stirred at room temperature for 6 h. The reaction mixture was quenched with water (30 ml) and extracted with DCM (2×50 ml). The combined organic layer was washed with brine (30 ml), dried over sodium sulphate and the solvent removed under reduced pressure to obtain 0.9 g of the crude title compound which was used in the next step without further purification. MS (ES+) m/z 537 [M+H]⁺

### Example 1

### 1-{2-methyl-[1,1′-biphenyl]-4-yl}-3-phenyl-1,3,5-triazinane-2,4,6-trione

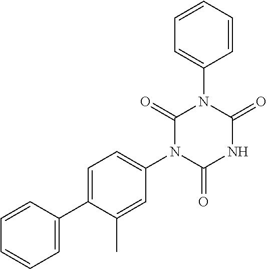 [SMILES: Cc1cc(-n2c(=O)[nH]c(=O)n(-c3ccccc3)c2=O)ccc1-c1ccccc1]

In a microwave vial previously equipped with a magnetic stirrer, 1-(2-methylbiphenyl-4-yl)-3-phenylurea (intermediate 1, 0.30 g, 0.9 mmol) and bromobenzene (6.0 ml) were added. The solution was cooled to 0° C. and ethoxy carbonyl isocyanate (0.45 g, 3.9 mmol) was added and the resulting reaction mixture was allowed to reach room temperature and then heated at 150° C. for 2 h in a microwave synthesizer. The solvent was evaporated under reduced pressure and the crude product was purified using preparative RP-HPLC (acetonitrile (40-100%) in water [0.1% formic acid]) to yield 0.035 g (9.5% yield) of the title compound. ¹H NMR (400 MHz, DMSO-d₆): δ12.05 (s, 1H), 7.52-7.44 (m, 5H), 7.42-7.37 (m, 5H), 7.33-7.28 (m, 3H), 2.27 (s, 3H); MS (ES−) m/z 370 [M−H]⁻.

### Example 2

### 1-[3-methyl-4-(piperidin-1-yl)phenyl]-3-phenyl-1,3,5-triazinane-2,4,6-trione

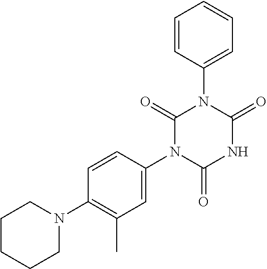 [SMILES: Cc1cc(-n2c(=O)[nH]c(=O)n(-c3ccccc3)c2=O)ccc1N1CCCCC1]

1-[3-methyl-4-(piperidin-1-yl)phenyl]-3-phenylurea (Intermediate 2, 0.35 g, 1.1 mmol) in bromobenzene (3.5 ml) was added to a microwave tube previously equipped with a magnetic stirrer and nitrogen balloon. The solution was cooled to 0° C. and ethoxy carbonyl isocyanate (0.52 g, 4.5 mmol) was added and resulting reaction mixture was allowed to reach room temperature and then heated at 150° C. for 3 h in a microwave synthesizer. The solvent was evaporated under reduced pressure and the crude product was purified using preparative RP-HPLC (15-100% acetonitrile in water [0.1% Formic acid]) to yield 0.010 g (2.3% yield) of the title compound. ¹H NMR (400 MHz, DMSO-d₆): δ11.97 (s, 1H), 7.49-7.36 (m, 5H), 7.15-7.01 (m, 3H), 2.82 (t, J=5.1 Hz, 4H), 2.25 (s, 3H), 1.66-1.54 (m, 6H); MS (ES−) m/z 377 [M−H]⁻.

### Example 3

### 1-[3-methyl-4-(propan-2-yl)phenyl]-3-phenyl-1,3,5-triazinane-2,4,6-trione

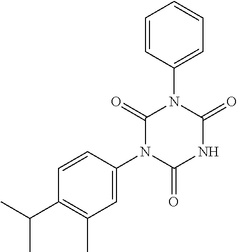 [SMILES: Cc1cc(-n2c(=O)[nH]c(=O)n(-c3ccccc3)c2=O)ccc1C(C)C]

In a microwave vial, previously equipped with a magnetic stirrer and nitrogen balloon, was added 1-[3-methyl-4-(propan-2-yl)phenyl]-3-phenylurea (Intermediate 4, 0.30 g, 1.1 mmol) in bromobenzene (6 ml). The solution was cooled to 0° C. and ethoxy carbonyl isocyanate (0.51 g, 4.4 mmol) was added and resulting reaction mixture was allowed to reach room temperature and heated at 150° C. for 3 h in a microwave synthesizer. The solvent was evaporated under reduced pressure and the crude product was purified by preparative RP-HPLC (30-100% acetonitrile in water [0.1% formic acid]) to yield 0.028 g (7% yield) of the title compound. ¹H NMR (400 MHz, DMSO-d₆): δ11.98 (s, 1H), 7.47-7.32 (m, 6H), 7.15-7.12 (m, 2H), 3.12-3.09 (m, 1H), 2.32 (s, 3H), 1.19 (d, J=6.8 Hz, 6H); MS (ES−) m/z 336 [M−H]⁻.

### Example 4

### 1-[3-methyl-4-(5-methylfuran-2-yl)phenyl]-3-phenyl-1,3,5-triazinane-2,4,6-trione

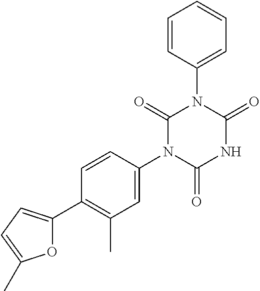 [SMILES: Cc1ccc(-c2ccc(-n3c(=O)[nH]c(=O)n(-c4ccccc4)c3=O)cc2C)o1]

1-[3-methyl-4-(5-methylfuran-2-yl)phenyl]-3-phenylurea (Intermediate 5, 0.40 g, 1.3 mmol) in bromobenzene (8.0 ml) was added to a microwave tube previously equipped with a magnetic stirrer and nitrogen balloon. The solution was cooled to 0° C. and to it ethoxy carbonyl isocyanate (0.60 g, 5.2 mmol) was added and resulting reaction mixture was allowed to reach room temperature and heated at 150° C. for 3 h in a microwave synthesizer. The solvent was evaporated under reduced pressure and the crude product was purified by preparative RP-HPLC (20-100% acetonitrile in water [0.1% formic acid]) to yield 0.022 g (4.5% yield) of the title compound. ¹H NMR (400 MHz, DMSO-d₆): δ12.04 (s, 1H), 7.72 (d, J=8.4 Hz, 1H), 7.50-7.38 (m, 5H), 7.28-7.26 (m, 2H), 6.68 (d, J=3.2 Hz, 1H), 6.27 (d, J=2.0 Hz, 1H), 2.44 (s, 3H), 2.34 (s, 3H); MS (ES−) m/z 374 [M−H]⁻.

### Example 5

### 1-{4′-ethyl-2-methyl-[1,1′-biphenyl]-4-yl}-3-methyl-1,3,5-triazinane-2,4,6-trione

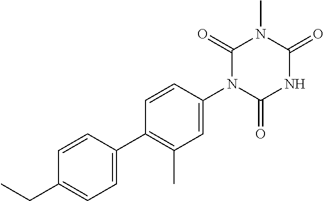 [SMILES: CCc1ccc(-c2ccc(-n3c(=O)[nH]c(=O)n(C)c3=O)cc2C)cc1]

1-(4′-ethyl-2-methylbiphenyl-4-yl)-3-methylurea (Intermediate 6, 0.30 g, 1.1 mmol) in bromobenzene (3.0 ml) was added to a microwave tube, previously equipped with a magnetic stirrer and nitrogen balloon, and cooled to 0° C. Ethoxy carbonyl isocyanate (0.51 g, 4.4 mmol) was added and resulting reaction mixture was allowed to reach room temperature and heated at 150° C. for 3 h in a microwave synthesizer. The solvent was evaporated under reduced pressure and the crude product was purified by preparative RP-HPLC (30-100% acetonitrile in water [0.1% formic acid]) to yield 0.035 g (9% yield) of the title compound. ¹H NMR (400 MHz, DMSO-d₆): δ11.87 (s, 1H), 7.32-7.17 (m, 7H), 3.17 (s, 3H), 2.68 (q, J=7.6 Hz, 2H), 2.26 (s, 3H), 1.25 (t, J=7.6 Hz, 3H); MS (ES−) m/z 336 [M−H]⁻

### Example 6

### 1-[3-methyl-4-(1H-pyrrol-1-yl)phenyl]-3-phenyl-1,3,5-triazinane-2,4,6-trione

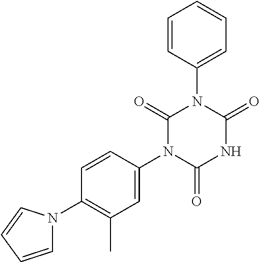 [SMILES: Cc1cc(-n2c(=O)[nH]c(=O)n(-c3ccccc3)c2=O)ccc1-n1cccc1]

1-[3-methyl-4-(1H-pyrrol-1-yl)phenyl]-3-phenylurea (Intermediate 7, 0.60 g, 2.0 mmol) in bromobenzene (6.0 ml) was added to a microwave tube previously equipped with a magnetic stirrer and nitrogen balloon. The solution was cooled to 0° C. and ethoxy carbonyl isocyanate (0.94 g, 8.2 mmol) was added and resulting reaction mixture was allowed to reach room temperature and heated at 150° C. for 3 h in a microwave synthesizer. The solvent was evaporated under reduced pressure and the crude product was purified by preparative RP-HPLC (25-100% acetonitrile in water [0.1% trifluoroacetic acid]) to yield 0.004 g (0.5% yield) of the title compound. ¹H NMR (400 MHz, DMSO-d₆): δ12.08 (s, 1H), 7.50-7.35 (m, 8H), 7.03 (s, 2H), 6.26 (s, 2H), 2.22 (s, 3H); MS (ES−) m/z 359 [M−H]⁻

### Example 7

### 1-methyl-3-{2-methyl-[1,1′-biphenyl]-4-yl}-1,3,5-triazinane-2,4,6-trione

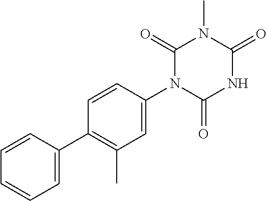 [SMILES: Cc1cc(-n2c(=O)[nH]c(=O)n(C)c2=O)ccc1-c1ccccc1]

In a microwave tube, previously equipped with a magnetic stirrer and nitrogen balloon, was added 1-methyl-3-(2-methylbiphenyl-4-yl)urea (Intermediate 8, 0.30 g, 1.2 mmol) in bromobenzene (3.0 ml). The solution was cooled to 0° C. and ethoxy carbonyl isocyanate (0.45 g, 4.9 mmol) was added. The resulting reaction mixture was allowed to reach room temperature and heated at 150° C. for 3 h in a microwave synthesizer. The solvent was removed under reduced pressure and the crude product was purified by preparative RP-HPLC (30-100% acetonitrile in water [0.1% formic acid]) to yield 0.016 g (4% yield) of the title compound. ¹H NMR (400 MHz, DMSO-d₆): δ11.90 (s, 1H), 7.51-7.47 (m, 2H), 7.42-7.40 (m, 3H), 7.31-7.21(m, 3H), 3.17 (s, 3H), 2.26 (s, 3H); MS (ES−) m/z 308 [M−H]⁻

### Example 8

### 1-[3-methyl-4-(1H-pyrazol-1-yl)phenyl]-3-phenyl-1,3,5-triazinane-2,4,6-trione

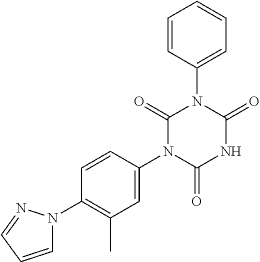 [SMILES: Cc1cc(-n2c(=O)[nH]c(=O)n(-c3ccccc3)c2=O)ccc1-n1cccn1]

Into a 30 ml microwave vial, previously equipped with a magnetic stirrer and nitrogen balloon was added 3-[3-methyl-4-(1H-pyrazol-1-yl)phenyl]-1-phenylurea (Intermediate 9, 0.3 g, 1.02 mmol) in Bromobenzene (3.0 ml). The mixture was cooled to 0° C. and Ethoxy carbonyl isocyanate (0.47 g, 4.10 mmol) was added and resulting reaction mixture was allowed to reach room temperature and then heated at 150° C. for 3 h in an Anton paar microwave synthesizer-300. The solvent was removed under reduced pressure to obtain the crude product that was purified by preparative RP-HPLC (50-100% MeOH in water [0.1% formic acid]) to yield 0.032 g (8% yield) of the title compound. ¹H NMR (400 MHz, DMSO-d₆: δ12.11 (s, 1H), 8.15 (d, J=2.00 Hz, 1H), 7.77 (s, 1H), 7.52-7.37 (m, 8H), 6.54 (s, 1H), 2.25 (s, 1H). MS (ES−) m/z 360 [M−H]⁻

### Example 9

### 1-[3-methyl-4-(3-methyl-1H-pyrrol-1-yl)phenyl]-3-(3-methylphenyl)-1,3,5-triazinane-2,4,6-trione

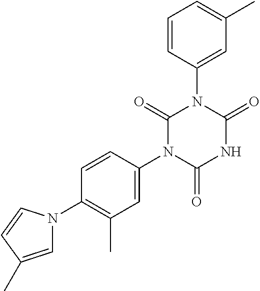 [SMILES: Cc1cccc(-n2c(=O)[nH]c(=O)n(-c3ccc(-n4ccc(C)c4)c(C)c3)c2=O)c1]

In a RBF previously equipped with a magnetic stirrer and nitrogen balloon was taken_3-[5-[3-methyl-4-(3-methyl-1H-pyrrol-1-yl)phenyl]-1-(3-methylphenyl)-4-(methylsulfanyl)-6-oxo-1,2,5,6-tetrahydro-1,3,5-triazin-2-ylidene]-1-(3-methylphenyl)urea (Intermediate 13, 0.450 g, 0.81 mmol) in 1,4-Dioxane (1.35 ml). To the solution 2M aq. HCl (2.7 ml) was added and the reaction mixture was heated to 100° C. and stirred for 2 h. The reaction mixture was allowed to reach room temperature and quenched with ice cold water (20 ml) and extracted with EtOAc (2×20 ml). The combined organic layer was washed with brine (20 ml), dried over sodium sulphate and solvent removed under reduced pressure. The crude product was purified by preparative RP-HPLC (30-100% Acetonitrile in water [0.1% formic acid]) to yield 0.031 g of the title compound. ¹H NMR (400 MHz, DMSO-d₆): δ12.03 (s, 1H), 7.38-7.30 (m, 4H), 7.25 (d, J=7.60 Hz, 1H), 7.19-7.17 (m, 2H), 6.89 (s, 1H), 6.77 (s, 1H), 6.08 (s, 1H), 2.38 (s, 3H), 2.23 (s, 3H), 2.09 (s, 3H); MS (ES−) m/z 387 [M−H]⁻

### Example 10

### 1-[4-(1H-indol-1-yl)-3-methylphenyl]-3-(3-methylphenyl)-1,3,5-triazinane-2,4,6-trione

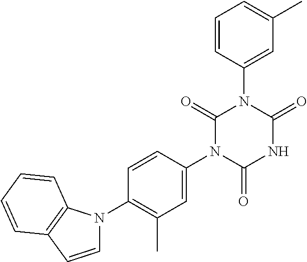 [SMILES: Cc1cccc(-n2c(=O)[nH]c(=O)n(-c3ccc(-n4ccc5ccccc54)c(C)c3)c2=O)c1]

In a microwave vial previously equipped with a magnetic stirrer and nitrogen balloon was taken 3-[4-(1H-indol-1-yl)-3-methylphenyl]-1-(3-methylphenyl)urea (Intermediate 16, 0.3 g, 0.845 mmol) in Bromobenzene (3 ml) and the mixture was cooled to 0° C. Ethoxy carbonyl isocyanate (0.388 g, 3.38 mmol) was added and resulting reaction mixture was allowed to reach 25° C. and heated at 150° C. for 3 h in Anton par microwave synthesizer-300. The solvent was removed under reduced pressure to obtain crude product that was purified by preparative RP-HPLC (20-100% Acetonitrile in water [5 mM ammonium bicarbonate +0.1% NH3]) to yield 0.015 g, (4%) of the title compound. ¹H NMR (400 MHz, DMSO-d6): δ12.04 (s, 1H), 7.67 (d, J=7.6 Hz, 1H), 7.54 (d, J=2.80 Hz, 1H), 7.48-7.45 (m, 2H), 7.41-7.35 (m, 2H), 7.25 -7.10 (m, 5H), 7.00 (d, J=7.6,1H), 6.69 (d, J=2.4,1H), 2.35 (s, 3H), 2.04 (s, 3H);); MS (ES−) m/z 423 [M−H]⁻

### Example 11

### 1-[3-methyl-4-(1H-pyrrol-1-yl)phenyl]-3-(3-methylphenyl)-1,3,5-triazinane-2,4,6-trione

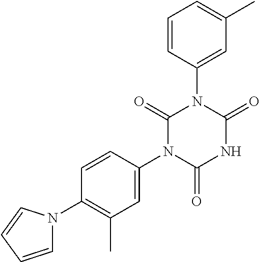 [SMILES: Cc1cccc(-n2c(=O)[nH]c(=O)n(-c3ccc(-n4cccc4)c(C)c3)c2=O)c1]

In a RBF previously equipped with a magnetic stirrer and nitrogen balloon was taken 3-[5-[3-methyl-4-(1H-pyrrol-1-yl)phenyl]-1-(3-methylphenyl)-4-(methylsulfanyl)-6-oxo-1,2,5,6-tetrahydro-1,3,5-triazin-2-ylidene]-1-(3-methylphenyl)urea (Intermediate 18, 0.8 g, 1.49 mmol) in 1,4-Dioxane (2.4 mi). To the solution 2M aq. HCI (4.8 ml) was added and the reaction mixture was heated to 100° C. and stirred for 2 h. The reaction mixture was allowed to reach room temperature and quenched with ice cold water (20 ml) and extracted with EtOAc (2×20 ml). The combined organic layer was washed with brine (20 ml), dried over sodium sulphate and the solvent removed under reduced pressure. The crude product was purified by preparative RP-HPLC (20-100% Acetonitrile in water [0.1% formic acid]) to yield 0.117 g of the title compound. ¹H NMR (400 MHz, DMSO-d₆: δ12.05 (s, 1H), 7.38-7.30 (m, 4H), 7.24 (d, J=7.60 Hz, 1H), 7.19-7.17 (m, 2H), 7.01 (s, 2H), 6.24 (s, 2H), 2.34 (s, 3H), 2.21 (s, 3H); MS (ES−) m/z 373 [M−H]⁻

### Biological Examples

In Vitro Assay

A high throughput cell-based screen has been used to identify positive modulators of TrkA and TrkB. The screen involves the use of cell-based assay overexpressing TrkA or TrkB. The purpose of the assay is to identify compounds that modulate neurotrophin signalling (Forsell et al 2012). The assay can be used in inhibitor mode using a high concentration of ligand, in modulator mode using an intermediate concentration and in agonist mode using a low concentration of ligand.

The assay uses Enzyme Fragment Complementation (EFC) technique, which is a proximity-based assay. Briefly, cells used in this assay over-express two fusion proteins, i.e. the receptor, which can be either of TrkA or TrkB, fused to a small peptide of beta-galactosidase and an adaptor protein, i.e. SHC1 (or any other Trk-adaptor protein) fused to the major part of beta-galactosidase. Ligand binding to the receptor induces phosphorylation of the intracellular domain and hence, recruitment of the adaptor protein to the receptor. The proximity between the small activating peptide on the receptor and the major part of beta-galactosidase on the adaptor protein leads to an active beta-galactosidase enzyme. The activation of the receptor is quantified by measuring the amount of active beta-galactosidase by its conversion of a non-luminescent substrate into a luminescent product.

U2OS-cells, over-expressing TrkA or TrkB, are plated in 96- or 384-well plates and incubated overnight. On the following day, test compound was pre-mixed with ligand (NGF) and the ligand-compound mixture is then added to the cells to yield a final ligand concentration of 10 ng/mL. After 3 hours of incubation at room temperature, the incubation is stopped by the addition of a beta-galactosidase substrate mixture containing detergents. The substrate mixture is incubated for 60 minutes at ambient temperature. The luminescence is thereafter read by the use of a plate reader.

Results

Data from these assays for representative compounds is shown in the Table below. The potency is expressed as EC50 (μM) for the individual receptors. The data indicate that the compounds of the invention are expected to possess useful therapeutic properties.

| Example | TrkA | TrkB |
|---|---|---|
| 1 | 0.18 | 0.09 |
| 2 | 1.05 | 1.27 |
| 3 | 7.05 | 7.9 |
| 4 | 6.77 | 2.08 |
| 5 | 7.2 | 3.0 |
| 6 | 0.24 | 0.25 |
| 7 | 6.71 | 20.6 |
| 8 | 5.56 | 6.22 |
| 11 | 0.10 | 0.07 |

1. A compound of formula I 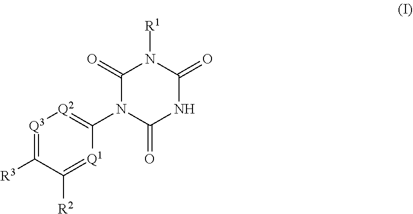
wherein
R¹ represents methyl; phenyl optionally substituted by one or more groups selected from halogen, —CN, —C(O)NR^{a1}R^{a2}, —NR^{a3}R^{a4}, 5-membered heteroaryl group, C₁₋₄ alkyl, C₁₋₄ alkoxy, C₁₋₄ alkoxy-C₁₋₄ alkyl or methylenedioxy, wherein the latter four groups are optionally substituted by one or more fluoro groups; or a 5-9-membered heteroaryl group optionally substituted by one or more groups selected from halogen, —CN, C₁₋₄ alkyl, C₁₋₄ alkoxy, C₁₋₄ alkoxy-C₁₋₄ alkyl or phenyl, wherein the latter four groups are optionally substituted by one or more fluoro groups;
Q¹, Q², and Q³ each represent —C(R⁴)— or —N—, wherein a maximum of two of Q¹ to Q³ represent —N—;
R⁴ represents H, halogen, C₁₋₄ alkyl C₁₋₄ alkoxy or C₁₋₄ alkoxy-C₁₋₄ alkyl, which latter three groups are optionally substituted by one or more fluoro groups;
R² represents halo; cyano; C₁₋₆ alkyl or C₁₋₆ alkoxy, which latter two groups are optionally substituted by one or more fluoro, ═O, hydroxy, —NR^{a5}R^{a6} or C₁₋₂ alkoxy groups;
R³ represents aryl optionally substituted by one or more G¹ groups; a 5-10-membered heteroaryl group optionally substituted by one or more G² groups; each of which two groups are optionally linked to the Q¹ to Q³-containing ring via an ethynylene group; a 4-10-membered heterocyclyl group optionally substituted by one or more G³ groups; C₁₋₆ alkyl, C₁₋₆ alkenyl, C₁₋₆ alkynyl, C₁₋₆ alkoxy or —N(C₁₋₆ alkyl)(C₁₋₆ alkyl), which latter five groups are optionally substituted by one or more G⁴ groups;
G¹ represents halogen; hydroxy; cyano; or C₁₋₄ alkyl, C₁₋₄ alkoxy or —NR^{a7}R^{a8}, wherein each of the latter three groups is optionally substituted by one or more fluoro groups;
G² represents halogen; hydroxy; cyano; or C₁₋₄ alkyl, C₁₋₄ alkoxy or —NR^{a9}R^{a10}wherein each of the latter three groups is optionally substituted by one or more fluoro groups; or phenyl or a 5-9-membered heteroaryl group, each of which two groups is optionally substituted by one or more groups selected from halo, —CN, C₁₋₂ alkyl, C₁₋₂ alkoxy or C₁₋₂ alkoxy-C₁₋₂ alkyl;
G³ represents halogen; hydroxy; cyano; or C₁₋₄ alkyl or C₁₋₄ alkoxy, wherein each of the latter two groups is optionally substituted by one or more fluoro groups; or phenyl or a 5-9-membered heteroaryl group, each of which two groups is optionally substituted by one or more groups selected from halo, —CN, C₁₋₂ alkyl or C₁₋₂ alkoxy; and
G⁴ represents halogen, ═O, hydroxy or C₁₋₂ alkoxy;
R^{a1}, R^{a2}, R^{a3}, R^{a4}, R^{a5}, R^{a6}, R^{a7}, R^{a8}, R^{a9} and R^{a10} each independently represent H or C₁₋₄ alkyl, which C₁₋₄ alkyl groups are optionally substituted by one or more fluoro groups; or
R^{a1} and R^{a2}, R^{a3} and R^{a4}, R^{a5} and R^{a6}, R^{a7} and R^{a8}, R^{a9} and R^{a10} may independently be joined together to form, together with the atom to which they are attached, a 4- to 6-membered heterocyclyl ring, which heterocyclyl ring optionally contains one further heteroatom selected from N, O or S. or a pharmaceutically-acceptable salt thereof.

2. A compound as claimed in claim 1, wherein R¹ represents methyl; phenyl optionally substituted by one or more groups selected from halogen, C₁₋₄ alkyl, C₁₋₄ alkoxy, or methylenedioxy, wherein the latter three groups are optionally substituted by one or more fluoro groups; or a 5-9-membered heteroaryl group optionally substituted by one or more groups selected from C₁₋₄ alkyl, C₁₋₄ alkoxy and phenyl, each of which groups is optionally substituted by one or more fluoro groups.

3. A compound as claimed in claim 1, wherein R¹ represents methyl; phenyl optionally substituted by one or more groups selected from fluoro, chloro, bromo, C₁₋₂ alkyl, C₁₋₂ alkoxy and methylenedioxy, wherein the latter three groups are optionally substituted by one or more fluoro groups; thiophenyl, thiazolyl, pyrazolyl, pyridinyl, benzofuranyl or indolyl each of which is optionally substituted by one or more groups selected from C₁₋₂ alkyl, C₁₋₂ alkoxy and phenyl each of which may be optionally substituted by one or more fluoro groups; benzofuranyl or indolyl.

4. A compound as claimed in claim 3, wherein R¹ represents methyl; or phenyl optionally substituted by one group selected from methyl, methoxy, chloro, fluoro, —OCF₃ and methylenedioxy.

5. A compound as claimed in claim 4, wherein R¹ represents phenyl or tolyl.

6. A compound as claimed in claim 4, wherein R¹ represents methyl or phenyl.

7. A compound as claimed in claim 1, wherein R² represents halo; cyano; C₁₋₆ alkyl or C₁₋₆ alkoxy, which latter two groups are optionally substituted by one or more fluoro, ═O, hydroxy, —N(C₁₋₂ alkyl)₂ or C₁₋₂ alkoxy groups.

8. A compound as claimed in claim 1, wherein R² represents chloro, bromo, C₁₋₄ alkyl or C₁₋₄ alkoxy, which latter two groups are optionally substituted by one or more fluoro, ═O, hydroxy, —NMe₂ or methoxy groups.

9. A compound as claimed in claim 8, wherein R² represents C₁₋₂ alkyl optionally substituted by one or more fluoro groups.

10. A compound as claimed in claim 9, wherein R² represents methyl.

11. A compound as claimed in claim 1, wherein R³ represents phenyl optionally substituted by one or more G¹ groups; a 5-9-membered heteroaryl group optionally substituted by one or more G² groups; each of which two groups is optionally linked to the Q¹ to Q³-containing ring via an ethynylene group; a 4-7-membered heterocyclyl group optionally substituted by one or more G³ groups; C₁₋₄ alkyl, C₁₋₄ alkenyl, C₁₋₄ alkynyl, C₁₋₄ alkoxy or —N(C₁₋₄ alkyl)(C₁₋₄ alkyl), which latter five groups are optionally substituted by one or more G⁴ groups, wherein the G¹, G², G³ and G⁴ groups are as defined in claim 1.

12. A compound as claimed in claim 11, wherein R³ represents phenyl optionally substituted by one or more G¹ groups; a 5-9-membered heteroaryl group selected from pyrrolyl, pyrazolyl, imidazolyl, triazolyl, tetrazolyl, oxazolyl, oxadiazolyl, thiazolyl, thiadiazolyl, thiophenyl furanyl, pyridinyl, pyridazinyl, pyrimidinyl, pyrazinyl, triazinyl, indolyl, benzimidazolyl, benzofuranyl, benzothiophenyl, azaindolyl and azabenzofuranyl each of which heteroaryl groups is optionally substituted by one or more G² groups; a heterocyclyl group selected from azetidinyl, piperidinyl, piperizinyl, morpholinyl, pyrrolidinyl, pyrazolidinyl, imidazolidinyl and imidazolinyl each of which heterocyclyl groups is optionally substituted by one or more G³ groups; or C₁₋₄ alkyl optionally substituted by one or more G⁴ groups, wherein the G¹, G², G³ and G⁴ groups are as defined in claim 1.

13. A compound as claimed in claim 12, wherein R³ represents phenyl optionally substituted by one or more G¹ groups; a 5-membered heteroaryl group selected from pyrrolyl, pyrazolyl, imidazolyl, triazolyl, tetrazolyl and furanyl, each of which heteroaryl groups is optionally substituted by one or more G² groups; a heterocyclyl group selected from azetidinyl, piperidinyl, piperizinyl, morpholinyl, pyrrolidinyl, pyrazolidinyl, imidazolidinyl and imidazolinyl each of which heterocyclyl groups is optionally substituted by one or more G³ groups; or C₁₋₄ alkyl optionally substituted by one or more G⁴ groups, wherein the G¹, G², G³ and G⁴ groups are as defined in claim 1.

14. A compound as claimed in claim 1, wherein R³ represents phenyl optionally substituted by one or more G¹ groups; or a 5-9-membered heteroaryl group selected from pyrrolyl, pyrazolyl, imidazolyl, triazolyl, tetrazolyl, thiophenyl, furanyl and indolyl, each of which heteroaryl groups is optionally substituted by one or more G² groups, wherein the G¹ and G² groups are as defined in claim 1.

15. A compound as claimed in claim 14, wherein R³ represents phenyl optionally substituted by one or more G¹ groups; or a 5-9-membered heteroaryl group selected from pyrrolyl, pyrazolyl, furanyl and indolyl, each of which heteroaryl groups is optionally substituted by one or more G² groups, wherein the G¹ and G² groups are as defined in claim 1.

16. A compound as claimed in claim 15, wherein R³ represents a 5-9-membered heteroaryl group selected from pyrrolyl, pyrazolyl, furanyl and indolyl, each of which heteroaryl groups is optionally substituted by one or more G² groups, wherein the G² groups are as defined in claim 1.

17. A compound as claimed in claim 16, wherein R³ represents pyrollyl or indolyl each of which is optionally substitutied by one or more G² groups, wherein the G² groups are as defined in claim 1.

18. A compound as claimed in claim 1, wherein G¹ represents halogen; hydroxy; cyano; or C₁₋₄ alkyl, C₁₋₄ alkoxy or —N(C₁₋₄ alkyl)(C₁₋₄ alkyl), wherein each of the latter three groups is optionally substituted by one or more fluoro groups.

19. A compound as claimed in claim 1, wherein G¹ represents fluoro; chloro; bromo; C₁₋₂ alkyl or C₁₋₂ alkoxy, wherein each of the latter two groups is optionally substituted by one or more fluoro groups.

20. A compound as claimed in claim 19, wherein G¹ represents C₁₋₂ alkyl optionally substituted by one or more fluoro group.

21. A compound as claimed in claim 1, wherein G² represents halogen; hydroxy; cyano; or C₁₋₄ alkyl, C₁₋₄ alkoxy or —N(C₁₋₄ alkyl)(C₁₋₄ alkyl), wherein each of the latter three groups is optionally substituted by one or more fluoro groups; or phenyl or a 5-9-membered heteroaryl group, each of which two groups is optionally substituted by one or more groups selected from halo, —CN, C₁₋₂ alkyl or C₁₋₂ alkoxy.

22. A compound as claimed in claim 1, wherein G² represents fluoro; chloro; bromo; C₁₋₂ alkyl, C₁₋₂ alkoxy or —N(C₁₋₂ alkyl)(C₁₋₂ alkyl), wherein each of the latter three groups is optionally substituted by one or more fluoro groups; or phenyl or a 5-6-membered heteroaryl group, each of which two groups is optionally substituted by one or more groups selected from fluoro, chloro, methyl or methoxy groups.

23. A compound as claimed in claim 1, wherein G² represents C₁₋₂ alkyl optionally substituted by one or more fluoro groups.

24. A compound as claimed in claim 1, wherein G³ represents fluoro; chloro; bromo; ═O; C₁₋₂ alkyl or C₁₋₂ alkoxy, wherein each of the latter two groups is optionally substituted by one or more fluoro groups; or phenyl or a 5-6-membered heteroaryl group, each of which two groups is optionally substituted by one or more groups selected from fluoro, chloro, methyl or methoxy groups.

25. A compound as claimed in claim 24, wherein G³ represents ═O or C₁₋₂ alkyl optionally substituted by one or more fluoro groups.

26. A compound as claimed in claim 1, wherein G⁴ represents fluoro, chloro, bromo or a C₁₋₂ alkoxy group, wherein the C₁₋₂ alkoxy group is optionally substituted by one or more fluoro groups.

27. A compound as claimed in claim 1, wherein R³ represents 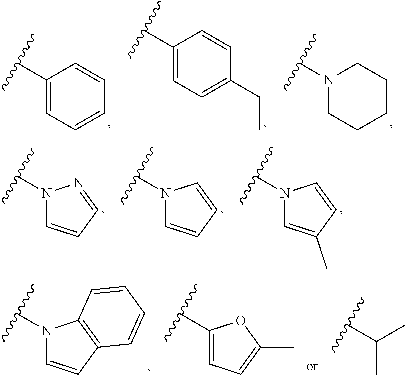
wherein [symbol] indicates the point of attachment to the phenyl ring.

28. A compound as claimed in claim 27, wherein R³ represents: 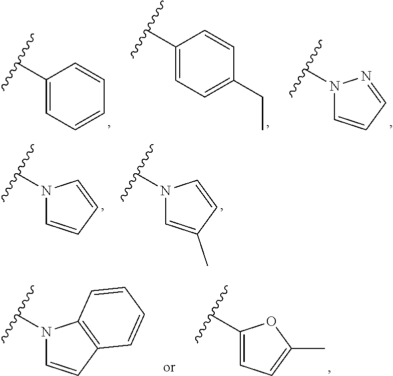
wherein [symbol] indicates the point of attachment to the phenyl ring.

29. A compound as claimed in claim 1, wherein one of Q¹, Q² andQ³ represents —N— and the others represent —C(R⁴)—.

30. A compound as claimed in claim 1, wherein Q¹, Q² and Q³ each represent —C(R⁴)—.

31. A compound as claimed in claim 1, wherein R⁴ represents H, halogen, C₁₋₄ alkyl or C₁₋₄ alkoxy, which latter two groups are optionally substituted by one or more fluoro groups.

32. A compound as claimed in claim 1, wherein each R⁴ group independently represents H, chloro, bromo, C₁₋₂ alkyl or C₁₋₂ alkoxy, which latter two groups are optionally substituted by one or more fluoro groups.

33. A compound as claimed in claim 32, wherein each R⁴ group represents H.

34. A compound as claimed in claim 1, wherein R^{a1}, R^{a2}, R^{a3}, R^{a4}, R^{a5}, R^{a6}, R^{a7}, R^{a8}, R^{a9} and R^{a10} each independently represent H or C₁₋₂ alkyl, which C₁₋₂ alkyl groups are optionally substituted by one or more fluoro groups.

35. A pharmaceutical composition comprising a compound as defined in claim 1, including pharmaceutically-acceptable salts thereof, in combination with one or more pharmaceutically-acceptable excipient.

36. (canceled)

37. (canceled)

38. A method of treating and/or preventing a disease characterised by impaired signalling of neurotrophins and/or other trophic factors, which comprises administering to a patient in need thereof a therapeutically effective amount of a compound as defined in claim 1.

39. (canceled)

40. The method claim 38, wherein the disease characterised by impaired signalling of neurotrophins and/or other trophic factors is selected from Alzheimer's disease, Lewy body dementia, frontotemporal dementia, HIV dementia, Huntington's disease, amyotrophic lateral sclerosis and other motor neurone diseases, Rett syndrome, epilepsy, Parkinson's disease and other parkinsonian disorders, disorders in which enhancement of nerve regeneration is beneficial, such as demyelinating diseases including multiple sclerosis, spinal cord injury, stroke, hypoxia, ischemia, brain injury including traumatic brain injury, mild cognitive impairment, dementia disorders, including dementia of mixed vascular and degenerative origin, presenile dementia, senile dementia and dementia associated with Parkinson's disease, progressive supranuclear palsy or corticobasal degeneration and cognitive dysfunction in schizophrenia, obesity, diabetes and metabolic syndrome, diabetic neuropathy including associated disorders such as osteoporosis, painful connective tissue disorders and tendon ruptures, Charcot Marie Tooth disease and its variants, nerve transplantation and its complications, motor neurone disease, peripheral nerve injury, genetic or acquired or traumatic hearing loss, blindness and posterior eye diseases, anterior eye diseases, depression, obesity, metabolic syndrome, pain, depression, schizophrenia, anxiety and constipation, including constipation in Parkinson's disease, slow-transit constipation and opioid-induced constipation.

41. The method of claim 40, wherein the disease characterised by impaired signalling of neurotrophins and/or other trophic factors is selected from the group consisting of Alzheimer's disease, Parkinson's disease, other Parkinsonian diseases, other tauopathies, Lewy body dementia, motorneurone disease, Pick disease, obesity, metabolic syndrome, diabetes, diabetic neuropathy, constipation and Rett Syndrome.

42. The method of claim 41, wherein the disease characterised by impaired signalling of neurotrophins and/or other trophic factors is selected from the group consisting of Alzheimer's disease, Parkinson's disease, cognitive dysfunction, depression, diabetic neuropathy, constipation and Rett syndrome.

43. The method of claim 42, wherein the disease characterised by impaired signalling of neurotrophins and/or other trophic factors is Alzheimer's disease.

44. A combination product comprising:
(I) a compound as defined in claim 1, or a pharmaceutically acceptable salt thereof; and
(II) one or more other therapeutic agent that is useful in the treatment or prevention of a disease characterised by impaired signalling of neurotrophins and/or other trophic factors, wherein each of components (I) and (II) is formulated in admixture, optionally with a pharmaceutically-acceptable excipient, such as a pharmaceutically-acceptable adjuvant diluent or carrier.

45. A kit-of-parts comprising:
(a) a pharmaceutical composition comprising a compound as defined in claim 1, or a pharmaceutically acceptable salt thereof, formulated in admixture with a pharmaceutically-acceptable excipient; and
(b) a pharmaceutical composition comprising one or more other therapeutic agent that is useful in the treatment or prevention of a disease characterised by impaired signalling of neurotrophins and/or other trophic factors, formulated in admixture with a pharmaceutically-acceptable excipient,
which components (a) and (b) are each provided in a form that is suitable for administration in conjunction with the other.

46. A process for the preparation of a compound as defined in claim 1, including a pharmaceutically-acceptable salt thereof, comprising the step of reacting a compound of formula II, 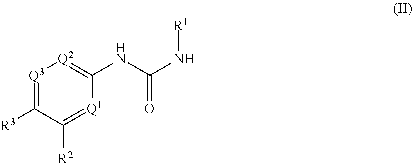
wherein R¹, R², R³, Q¹, Q² and Q³ are as defined in as above, with ethoxycarbonyl isocyanate; or
the step of reacting a compound of formula IX 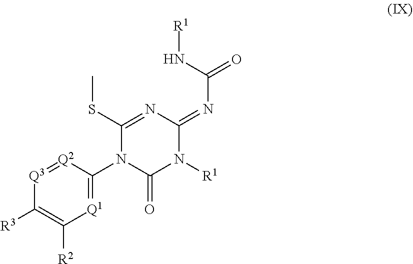
wherein R¹, R², R³, Q¹, Q² and Q³ are as defined as above, in the presence of a suitable acid.
